(19) United States
(12) Patent Application Publication
(10) Pub. No.: US 2024/0309006 A1
(43) Pub. Date: Sep. 19, 2024

(54) NOVEL BICYCLIC COMPOUNDS

(71) Applicant: PRISM Biolab Co., Ltd.

(72) Inventors: Hiroyuki KOUJI, Kanagawa (JP); Takenao ODAGAMI, Osaka (JP); Yoichiro HIROSE, Kanagawa (JP); Takashi TAKAHASHI, Kanagawa (JP); Hisashi MASUI, Nagoya-shi, Aichi (JP); Atsushi YOSHIMORI, Kanagawa (JP); Hajime TAKASHIMA, Kanagawa (JP); Jun OZAWA, Kanagawa (JP); Eiji HONDA, Kanagawa (JP)

(73) Assignee: PRISM Biolab Co., Ltd., Kanagawa (JP)

(21) Appl. No.: 18/273,064

(22) PCT Filed: Jan. 19, 2022
(86) PCT No.: PCT/JP2022/003469

§ 371 Date: Jul. 19, 2023

Related U.S. Application Data
(60) Provisional application No. 63/139,443, filed on Jan. 20, 2021.

(51) Int. Cl. C07D 487/04; A61K 31/4985; A61K 31/551

(52) U.S. Cl. CPC C07D 487/04; A61K 31/4985; A61K 31/551

(57) ABSTRACT

A compound of the formula (I): wherein each symbol is as defined in the DESCRIPTION. or a pharmaceutically acceptable salt thereof has a superior Notch signal transduction inhibitory action, and is useful for preventing or treating various diseases involving Notch signal transduction.

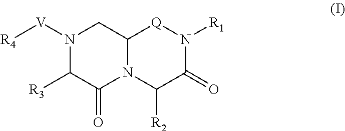

### TECHNICAL FIELD

The present invention relates to a novel bicyclic compound. More specifically, the present invention relates to a novel bicyclic compound having Notch inhibitory action.

### BACKGROUND ART

Notch signaling is an evolutionary conserved pathway that plays an integral role in development and tissue homeostasis in mammals. The Notch receptors and ligands contain single-pass transmembrane domains, are expressed on the cell surface and, for that reason, Notch signaling is particularly important in mediating communication between adjacent cells expressing the receptors and ligands. There are four known Notch receptors found in rodents and humans, termed Notch 1 to Notch 4. The Notch receptors are heterodimeric proteins composed of extracellular and intracellular domains that are initially synthesized as a single polypeptide. Receptor-ligand interaction triggers a series of proteolytic cleavages of the Notch receptor polypeptide in which γ-secretase activity is involved. γ-Secretase activity cleaves Notch intracellular domain from the internal side of the plasma membrane which translocates to the nucleus to form a transcription factor complex. Notch intracellular domain (NICD) is the active form of the protein. Various Notch signaling functions include proliferation, differentiation, apoptosis, angiogenesis, migration and self-renewal (Non-patent documents 1-3) .

In addition, NICD activates transcription of the target genes Hes1 and Hes5 by translocation into the nucleus and forming a stable complex with RBP-J and MAML, which are DNA binding proteins.

Therefore, a compound that can inhibit various Notch signal function can be a medicament useful for various diseases involving the function.

### Document List

### Non-Patent Documents

non-patent document 1: Bray, Nature Reviews Molecular Cell Biology, 7:678-689 (2006).
non-patent document 2: Fortini, Developmental Cell 16:633-647 (2009).
non-patent document 3: Ables, J. L. et al., Neurosci., 12: 269-283 (2011).

### SUMMARY OF THE INVENTION

### Problems to be Solved by the Invention

The present invention aims to provide a compound having a Notch inhibitory action and a medicament containing the compound and useful for various diseases.

### Means of Solving the Problems

The present inventors have conducted intensive studies in an attempt to solve the aforementioned problems and found that a compound having a particular structure shows a superior Notch signal transduction inhibitory action (hereinafter to be also referred to as Notch inhibitor), and completed the present invention.

That is, the present invention relates to the following.
[1] A compound represented by the following formula (I):

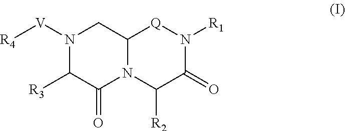

wherein
R₁ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl;
R₂ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl;
R₃ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl;
Q is —CH₂— or —CH₂CH₂—;
V is a bond, —CO—, —SO₂—, —NHCO—, or —OCO—; and
R₄ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl, or a pharmaceutically acceptable salt thereof.
[2] A compound of [1], which is represented by the following formula (II):

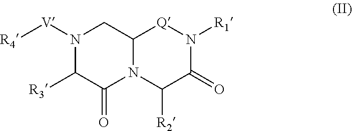

wherein
R₁′ is hydrogen, optionally substituted alkyl, optionally substituted heterocycloalkyl, or optionally substituted arylalkyl;
R₂′ is optionally substituted alkyl, or optionally substituted arylalkyl;
R₃′ is hydrogen, optionally substituted alkyl, or optionally substituted arylalkyl;
Q′ is —CH₂— or —CH₂CH₂—;
V′ is a bond, or —CO—; and
R4′ is hydrogen, optionally substituted alkyl, optionally substituted aryl, or optionally substituted arylalkyl, or a pharmaceutically acceptable salt thereof.
[3] A pharmaceutical composition comprising a compound of [1] or [2] or a pharmaceutically acceptable salt thereof, and optionally a pharmaceutically acceptable carrier or diluent.
[4] A pharmaceutical composition of [3], wherein the composition comprises an effective amount of the compound.
[5] A method of treating or preventing a disease involving Notch signal transduction comprising administering to a subject in need thereof a compound of [1] or [2] or a pharmaceutically acceptable salt thereof, or a composition of [3] or [4], in an amount effective to treat or prevent the disease.
[6] An agent for treating or preventing a disease involving Notch signal transduction comprising a compound of [1] or [2] or a pharmaceutically acceptable salt thereof.
[7] A compound of [1] or [2] or a pharmaceutically acceptable salt thereof, or a composition of [3] or [4] for the use as a medicament for treating or preventing a disease involving Notch signal transduction.

### EFFECT OF THE INVENTION

The compound of the formula (I) of the present invention inhibits Notch signal transduction and thus can be used for treating various diseases involving Notch signal transduction.

### BRIEF DESCRIPTION OF THE DRAWINGS

FIG. 1 shows a ¹H NMR (400 MHZ, CDCl₃) data of 2_X01′a.

FIG. 2 shows a ¹H NMR (400 MHZ, CDCl₃) data of 7X01Y01a.

FIG. 3 shows a ¹H NMR (400 MHZ, CDCl₃) data of 6_X01″Y04′a.

### DESCRIPTION OF EMBODIMENTS

### Definition

Unless otherwise stated, the following terms used in the specification and claims shall have the following meanings for the purposes of this Application.

“Lower”, unless indicated otherwise, means that the number of the carbon atoms constituting the given radicals is between one and six.

“Optionally substituted”, unless otherwise stated, means that a given radical may consist of only hydrogen substituents through available valencies or may further comprise one or more non-hydrogen substituents through available valencies. In general, a non-hydrogen substituent may be any substituent that may be bound to an atom of the given radical that is specified to be substituted. Examples of substituents include, but are not limited to, —R₆, —OR₆, —COR₆, —COOR₆, —OCOR₆, —CONR₆R₇, —NR₆R₇, —NR₇COR₆, —NR₇COOR₆, —SR₆, —SO₂R₆, —SO₂NR₆R₇, —SO₂OR₆, —OSO₂R₆, —NHC(NHR₆)NR₇, —NHC(NH₂)NH, —OPO(OH)₂, —OPO(ONa)₂, —CN, —NO₂, halogen and methylenedioxy, wherein R⁶ and R⁷ is independently selected from hydrogen, linear or branched chain, cyclic or noncyclic, substituted or unsubstituted, alkyl chain, aryl, heteroaryl, arylalkyl and heteroarylalkyl moieties.

“Halogen” means fluorine, chlorine, bromine or iodine. “Halo” means fluoro, chloro, bromo or iodo.

“Alkyl” means a linear or branched, saturated, aliphatic radical having a chain of carbon atoms. C_{X-Y} alkyl is typically used where X and Y indicate the number of carbon atoms in the chain. The number of carbon atoms in the chain is preferably 1 to 10 (C₁₋₁₀), more preferably 1 to 6 (C₁₋₆), further preferably 1 to 4 (C₁₋₄). Non-exclusive examples of alkyl include methyl, ethyl, propyl, isopropyl, butyl, sec-butyl, isobutyl, tert-butyl, pentyl, isopentyl, neopentyl, tert-pentyl, hexyl, isohexyl, and the like.

“Alkenyl” means a linear or branched, carbon chain that contains at least one carbon-carbon double bond. C_{X-Y} alkenyl is typically used where X and Y indicate the number of carbon atoms in the chain. The number of carbon atoms in the chain is preferably 2 to 10 (C₂₋₁₀) , more preferably 2 to 6 (C₂₋₆). Non-exclusive examples of alkenyl include ethenyl (vinyl), allyl, isopropenyl, 2-methylallyl, 1-pentenyl, hexenyl, heptenyl, 1-propenyl, 2-butenyl, 2-methyl-2-butenyl, and the like.

“Alkynyl” means a linear or branched, carbon chain that contains at least one carbon-carbon triple bond. C_{X-Y} alkynyl is typically used where X and Y indicate the number of carbon atoms in the chain. The number of carbon atoms in the chain is preferably 2 to 10 (C₂₋₁₀), more preferably 2 to 6 (C₂₋₆). Non-exclusive examples of alkynyl include ethynyl, propargyl, 3-methyl-1-pentynyl, 2-heptynyl and the like.

“Alkylene”, unless indicated otherwise, means a linear or branched, saturated, aliphatic, polyvalent carbon chain. C_{X-Y} alkylene is typically used where X and Y indicate the number of carbon atoms in the chain. The number of carbon atoms in the chain is preferably 1 to 10 (C₁₋₁₀), more preferably 1 to 6 (C₁₋₆). Non-exclusive examples of alkylene include methylene (—CH₂—) , ethylene (—CH₂CH₂—), methylmethylene (—CH(CH₃)—), 1,2-propylene (—CH₂CH(CH₃)—), 1,3-propylene (—CH₂CH₂CH₂—), 1,2-butylene (—CH₂CH(CH₂CH₃)—), 1,3-butylene (—CH₂CH₂CH(CH₃)—), 1,4-butylene (—CH₂CH₂CH₂CH₂—), 2-methyltetramethylene (—CH₂CH(CH₃)CH₂CH₂—) , pentamethylene (—CH₂CH₂CH₂CH₂CH₂—), 1,2,3-propanetriyl, 1,3,3-propanetriyl and the like.

“Heteroatom” refers to an atom that is not a carbon atom and a hydrogen atom. Particular examples of heteroatoms include, but are not limited to nitrogen, oxygen, and sulfur.

“Aryl” means a monocyclic or polycyclic radical wherein each ring is aromatic or when fused with one or more rings form an aromatic ring. C_{X-Y} aryl is typically used where X and Y indicate the number of carbon atoms in the ring assembly. The number of carbon atoms in the ring is preferably 6 to 14 (C₆₋₁₄) , more preferably 6 to 10 (C₆₋₁₀). Non-exclusive examples of aryl include phenyl, naphthyl, indenyl, azulenyl, biphenyl, fluorenyl, anthracenyl, phenalenyl and the like. “Aryl” may partially be hydrogenated. Non-exclusive examples of partially hydrogenated aryl include tetrahydronaphthyl, indanyl and the like.

“Heteroaryl” means a monocyclic or polycyclic aromatic radical wherein at least one ring atom is a heteroatom and the remaining ring atoms are carbon. “X-Y membered heteroaryl” is typically used where X and Y indicate the number of carbon atoms and heteroatoms in the ring assembly. The number of carbon atoms and heteroatoms in the ring is preferably 5 to 14, more preferably 5 to 10. Monocyclic heteroaryl groups include, but are not limited to, cyclic aromatic groups having five or six ring atoms, wherein at least one ring atom is a heteroatom and the remaining ring atoms are carbon. The nitrogen atoms can be optionally quaternerized and the sulfur atoms can be optionally oxidized. Non-exclusive examples of monocyclic heteroaryl group of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrrole, thiazole, 1,3,4-thiadiazole, triazole and tetrazole. “Heteroaryl” also includes, but is not limited to, bicyclic or tricyclic rings, wherein the heteroaryl ring is fused to one or two rings independently selected from the group consisting of an aryl ring, a cycloalkyl ring, and another monocyclic heteroaryl or heterocycloalkyl ring. Non-exclusive examples of bicyclic or tricyclic heteroaryl include, but are not limited to, those derived from benzofuran (ex. benzo[b]furan), benzothiophene (ex. benzo[b]thiophene), benzimidazole, benzotriazine (ex. benzo[e][1,2,4]triazine, benzo[d][1,2,3]triazine), pridopyrimidine (ex. pyrido[4,3-d]pyrimidine, pyrido[3,4-d]pyrimidine, pyrido[3,2-d]pyrimidine, pyrido[2,3-d]pyrimidine), pyridopyrazine (ex. pyrido[3,4-b]pyrazine, pyrido[2,3-b]pyrazine), pyridopyridazine (ex. pyrido[2,3-c]pyridazine, pyrido[3,4-c]pyridazine, pyrido[4,3-c]pyridazine, pyrido[3,2-c]pyridazine), pyridotriazine (ex. pyrido[2,3-d][1,2,3]triazine, pyrido[3,4-d][1,2,3]triazine, pyrido[4,3-d][1,2,3]triazine, pyrido[3,2-d][1,2,3]triazine, pyrido[3,4-e][1,2,4]triazine, pyrido[3,2-e][1,2,4]triazine), benzothiadiazole (ex. benzo[c][1,2,5]thiadiazole), furopyridine (ex. furo[3,2-b]pyridine, furo[3,2-c]pyridine, furo[2,3-c]pyridine, furo[2,3-b]pyridine), oxazolopyridine (ex. oxazolo[4,5-b]pyridine, oxazolo[4, 5-c]pyridine, oxazolo[5, 4-c]pyridine, oxazolo[5,4-b]pyridine), thiazolopyridine (ex. thiazolo[4,5-b]pyridine, thiazolo[4,5-c]pyridine, thiazolo[5,4-c]pyridine, thiazolo[5,4-b]pyridine), imidazopyridine (ex. imidazo[1,2-a]pyridine, imidazo[4,5-c]pyridine, imidazo[1,5-a]pyridine), quinazoline, thienopyridine (ex. thieno[2,3-c]pyridine, thieno[3,2-b]pyridine, thieno[2,3-b]pyridine), indolizine, quinoline, isoquinoline, phthalazine, quinoxaline, cinnoline, naphthyridine, quinolizine, indole, isoindole, indazole, indoline, benzoxazole, benzopyrazole, benzothiazole, pyrazolopyridine (ex. pyrazolo[1,5-a]pyridine), imidazopyrimidine (ex. imidazo[1,2-a]pyrimidine, imidazo[1,2-c]pyrimidine, imidazo[1,5-a]pyrimidine, imidazo[1,5-c]pyrimidine), pyrrolopyridine (ex. pyrrolo[2,3-b]pyridine, pyrrolo[2,3-c]pyridine, pyrrolo[3,2-c]pyridine, pyrrolo[3,2-b]pyridine), pyrrolopyrimidine (ex. pyrrolo[2,3-d]pyrimidine, pyrrolo[3,2-d]pyrimidine, pyrrolo[1,2-c]pyrimidine, pyrrolo[1,2-a]pyrimidine), pyrrolopyrazine (ex. pyrrolo[2,3-b]pyrazine, pyrrolo[1,2-a]pyrazine), pyrrolopyridazine (ex. pyrrolo[1,2-b]pyridazine), triazopyridine (ex. triazo[1,5-a]pyridine), pteridine, purine, carbazole, acridine, perimidine, 1,10-phenenthroline, phenoxathiin, phenoxazine, phenothiazine, phenazine and the like. The bicyclic or tricyclic heteroaryl rings can be attached to the parent molecule through either the heteroaryl group itself or the aryl, cycloalkyl, or heterocycloalkyl group to which it is fused.

“Cycloalkyl” means a non-aromatic, saturated or partially unsaturated, monocyclic, fused bicyclic or bridged polycyclic ring radical. C_{X-Y} cycloalkyl is typically used where X and Y indicate the number of carbon atoms in the ring assembly. The number of carbon atoms in the ring is preferably 3 to 10 (C₃₋₁₀), more preferably 3 to 8 (C₃₋₈). Non-exclusive examples of cycloalkyl include cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, bicyclo[2.2. 1]hept-1-yl, and the like.

“Heterocycloalkyl” means cycloalkyl, as defined in this Application, provided that one or more of the atoms forming the ring is a heteroatom selected, independently from N, O, or S. “X-Y membered heterocycloalkyl” is typically used where X and Y indicate the number of carbon atoms and heteroatoms in the ring assembly. The number of carbon atoms and heteroatoms in the ring is preferably 3 to 10, more preferably 3 to 8. Non-exclusive examples of heterocycloalkyl include piperidyl, 4-morpholyl, 4-piperazinyl, pyrrolidinyl, perhydropyrrolidinyl, 1,4-diazaperhydroepinyl, 1,3-dioxanyl, 1,4-dioxanyl, and the like.

Moreover, the above-mentioned definitions can apply to groups wherein the above-mentioned substituents are connected. For example, “arylalkyl” means linear or branched alkyl group which is substituted by one or more aryl groups, such as benzyl, 1-phenylethyl, 2-phenylethyl, 3-phenylpropyl, 1-naphthylmethyl, 2-naphthylmethyl and the like. “Heteroarylalkyl” means linear or branched alkyl group which is substituted by one or more heteroaryl groups.

“Cycloalkylalkyl” means linear or branched alkyl group which is substituted by one or more cycloalkyl group (e.g., cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, bicyclo[2.2.1]hept-1-yl).

“Heterocycloalkylalkyl” means linear or branched alkyl group which is substituted by one or more heterocycloalkyl groups.

“Monocyclic ring” as used herein refers to a monocyclic, saturated or unsaturated carbocyclic ring or a monocyclic, saturated or unsaturated heterocyclic ring. “X-membered monocyclic ring” is typically used where X indicate the number of carbon atoms and heteroatoms in the ring assembly. The number of carbon atoms and heteroatoms in the ring is preferably 4 to 7, more preferably 5 or 6. “Monocyclic heterocyclic ring” means a monocyclic, aromatic or nonaromatic ring wherein at least one ring atom is a heteroatom (preferably S, N or O) and the remaining ring atoms are carbon. The nitrogen atoms can be optionally quaternized and the sulfur atoms can be optionally oxidized.

Non-exclusive examples of monocyclic saturated carbocyclic ring include cyclopropane, cyclobutane, cyclopentane, cyclohexane, cycloheptane and the like.

Non-exclusive examples of monocyclic unsaturated carbocyclic ring include cyclopropene, cyclobutene, cyclopentene, cyclohexene, cycloheptene, cyclopentadiene, benzene, and the like.

Non-exclusive examples of monocyclic saturated heterocyclic ring include pyrrolidine, piperidine, morpholine, piperazine, 1,3-dioxane, 1,4-dioxane and the like.

Non-exclusive examples of monocyclic unsaturated heterocyclic ring include pyrazole, dihydro-pyrrole, pyrrole, dihydro-pyrazole, imidazole, thiophene, thiazole, isothiazole, thiadiazole, furan, oxazole, isoxazole, oxadiazole, pyridine, pyridazine, pyrimidine, pyrazine, triazine and the like.

“Spiro ring” as used herein refers to saturated or unsaturated cycloalkane or saturated or unsaturated heterocycloalkane.

“Cycloalkane” means a non-aromatic, saturated or partially unsaturated, monocyclic, fused bicyclic or bridged polycyclic ring. C_{X-Y} cycloalkane is typically used where X and Y indicate the number of carbon atoms in the ring assembly. The number of carbon atoms in the ring is preferably 3 to 10 (C₃₋₁₀), more preferably 3 to 8 (C₃₋₈). Non-exclusive examples of cycloalkane include cyclopropane, cyclobutane, cyclopentane, cyclohexane, cycloheptane, cyclooctane and the like.

“Heterocycloalkane” means cycloalkane, as defined in this Application, provided that one or more of the atoms forming the ring is a heteroatom selected, independently from N, O, and S. “X-Y membered heterocycloalkane” is typically used where X and Y indicate the number of carbon atoms and heteroatoms in the ring assembly. The number of carbon atoms and heteroatoms in the ring is preferably 3 to 10, more preferably 3 to 8. Non-exclusive examples of heterocycloalkane include piperidine, morpholine, piperazine, pyrrolidine, perhydropyrrolizine, tetrahydrofuran, tetrahydropyran, 1,3-dioxane, 1,4-dioxane and the like.

“Derivatives” mean a compound differing from another compound by a structural modification, for example by replacement of one atom or a group of atoms or a functional group with another atom or group atoms or functional group. “Protected derivatives” mean derivatives of compound in which a reactive site or sites are blocked with protecting groups. A comprehensive list of suitable protecting groups can be found in T. W. Greene, Protecting Groups in Organic Synthesis, 5th edition, John Wiley & Sons, Inc.2014.

The compounds of the present invention may include these derivatives or protected derivatives.

“Isomers” mean any compound having identical molecular formulae but differing in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality. A mixture of the two enantiomeric forms is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). The compounds of the present invention may include these isomers.

“Animal” includes humans, non-human mammals (e.g., mice, rats, dogs, cats, rabbits, cattle, horses, sheep, goats, swine, deer, and the like) and non-mammals (e.g., birds, and the like).

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salt” or “salt” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, trifluoroacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Amount effective to treat” means that amount which, when administered to an animal for treating a disease, is sufficient to effect such treatment for the disease.

“Amount effective to prevent” means that amount which, when administered to an animal for preventing a disease, is sufficient to effect such prophylaxis for the disease.

“Effective amount” equals to “amount effective to treat” and “amount effective to prevent”.

“Treatment” or “treat” means any administration of a compound of the present invention and includes:
(1) preventing the disease from occurring in an animal which may be predisposed to the disease but does not yet experience or display the pathology or symptomatology of the disease,
(2) inhibiting the disease in an animal that is experiencing or displaying the pathology or symptomatology of the diseased (i.e., arresting further development of the pathology and/or symptomatology), or
(3) ameliorating the disease in an animal that is experiencing or displaying the pathology or symptomatology of the diseased (i.e., reversing the pathology and/or symptomatology).

It is noted in regard to all of the definitions provided herein that the definitions should be interpreted as being open ended in the sense that further substituents beyond those specified may be included.

In another embodiment of the formula (I), a compound having the following formula (II):

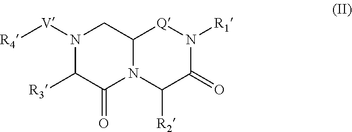

wherein
R₁′ is hydrogen, optionally substituted alkyl, optionally substituted heterocycloalkyl, or optionally substituted arylalkyl;
R₂′ is optionally substituted alkyl, or optionally substituted arylalkyl;
R₃′ is hydrogen, optionally substituted alkyl, or optionally substituted arylalkyl;
Q′ is —CH₂— or —CH₂CH₂—;
V′ is a bond, or —CO—; and
R₄′ is hydrogen, optionally substituted alkyl, optionally substituted aryl, or optionally substituted arylalkyl, or a pharmaceutically acceptable salt thereof is disclosed.

In one embodiment of the formula (I), R₁ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl and the like.

Examples of optionally substituted alkenyl group including ethenyl, allyl, 1-propenyl, 2-methylallyl and the like.

Examples of optionally substituted alkynyl group include ethynyl, 1-propynyl, and the like.

Examples of optionally substituted aryl and optionally substituted heteroaryl include biphenyl, phenyl, pyridyl, pyrimidyl, pyridazinyl, pyrazinyl, triazinyl, pyrrolyl, thienyl, furyl, thiazolyl, oxazolyl, imidazolyl, tetrahydronaphthyl, naphthyl, quinolinyl, isoquinolinyl, quinazolinyl, quinoxalinyl, cinnolinyl, naphthyridinyl, benzotriazinyl, indenyl, pyridopyrimidinyl, pyridopyrazinyl, pyridopyridazinyl, pyridotriazinyl, benzofuryl, benzothienyl, indolyl, indazolyl, benzoxazolyl, benzimidazolyl, benzothiazolyl, benzothiadiazolyl, furopyridinyl, thienopyridinyl, pyrropyridinyl, oxazolopyridinyl, thiazolopyridinyl, imidazopyridinyl and the like.

Examples of optionally substituted cycloalkyl include cyclopropyl, cyclobutyl, cyclopentyl, cyclohexyl, cycloheptyl, adamantyl and the like.

Examples of optionally substituted heterocycloalkyl include piperidyl, 4-morpholyl, 4-piperazinyl, pyrrolidinyl, perhydropyrrolidinyl, 1,4-diazaperhydroepinyl, 1,3-dioxanyl, 1,4-dioxanyl, benzyloxycarbonylpiperidinyl and the like.

Examples of the optionally substituted arylalkyl group include unsubstituted arylalkyl or arylalkyl having an alkyl group such as benzyl, α-methylbenzyl, phenethyl, α-methylphenethyl, α, α-dimethylbenzyl, α,α-dimethylphenethyl, 4-methylphenethyl, 4-methylbenzyl, 4-isopropylbenzyl and the like; arylalkyl having an aryl group or an arylalkyl group such as 4-benzylbenzyl, 4-phenethylbenzyl, 4-phenylbenzyl and the like; arylalkyl having a substituted oxy group such as 4-methoxybenzyl, 4-n-tetradecyloxybenzyl, 4-n-heptadecyloxybenzyl, 3,4-dimethoxybenzyl, 4-methoxymethylbenzyl, 4-vinyloxymethylbenzyl, 4-benzyloxybenzyl, 4-phenethyloxybenzyl and the like; arylalkyl having a hydroxyl group such as 4-hydroxybenzyl, 4-hydroxy-3-methoxybenzyl, hydroxyphenethyl and the like; arylalkyl having a halogen atom such as 4-fluorobenzyl, 3-chlorobenzyl, 3,4-dichlorobenzyl and the like; 2-furfuryl, diphenylmethyl, 1-naphthylmethyl, 2-naphthylmethyl and the like.

Examples of the optionally substituted heteroarylalkyl group include 2-pyridylmethyl, 3-pyridylmethyl, 2-pyrimidinylmethyl, 5-pyrimidinylmethyl, 3-pyridazinylmethyl, 2-indolylmethyl, 5-indolylmethyl, 2-benzofuranylmethyl, 5-indolylmethyl, 2-benzothienylmethyl, 5-benzothienylmethyl, 6-fluoro-2-benzofuranylmethyl, 6-chloro-2-benzofuranylmethyl, 6-methoxy-2-benzofuranylmethyl, 6-fluoro-2-benzothienylmethyl, 6-chloro-2-benzothienylmethyl, 6-methoxy-2-benzothienylmethyl and 6-phenyl-3-pyridazinylmethyl and the like.

Examples of the optionally substituted cycloalkylalkyl group include cyclopropylmethyl, fluorocyclopropylmethyl, chlorocyclopropylmethyl, bromocyclopropylmethyl, iodocyclopropylmethyl, methylcyclopropylmethyl, 1,1-dimethylcyclopropylmethyl, 1,2-dimethylcyclopropylmethyl, hydroxycyclopropylmethyl, methoxycyclopropylmethyl, ethoxycyclopropylmethyl, methoxycarbonylcyclopropylmethyl, methylcarbamoylcyclopropylmethyl, cyclopropylethyl, cyclohexylmethyl, cyclopropylhexyl and the like.

Examples of the optionally substituted heterocycloalkylalkyl group include (2-tetrahydrofuryl) methyl, (2-tetrahydrothiofuranyl) methyl and the like.

In another embodiment of the formula (I), R₁ is hydrogen, optionally substituted alkyl (e.g., isopentyl), optionally substituted heterocycloalkyl (e.g., benzyloxycarbonylpiperidinyl), or optionally substituted arylalkyl (e.g., phenethyl, hydroxyphenethyl).

In one embodiment of the formula (I), R₂ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl, aminocarbonylmethyl, aminocarbonylethyl, aminocarbonylpropyl and the like.

Examples of optionally substituted alkenyl group including ethenyl, allyl, 1-propenyl, 2-methylallyl and the like.

Examples of optionally substituted alkynyl group include ethynyl, 1-propynyl, and the like.

Examples of optionally substituted aryl and optionally substituted heteroaryl include biphenyl, phenyl, pyridyl, pyrimidyl, pyridazinyl, pyrazinyl, triazinyl, pyrrolyl, thienyl, furyl, thiazolyl, oxazolyl, imidazolyl, tetrahydronaphthyl, naphthyl, quinolinyl, isoquinolinyl, quinazolinyl, quinoxalinyl, cinnolinyl, naphthyridinyl, benzotriazinyl, indenyl, pyridopyrimidinyl, pyridopyrazinyl, pyridopyridazinyl, pyridotriazinyl, benzofuryl, benzothienyl, indolyl, indazolyl, benzoxazolyl, benzimidazolyl, benzothiazolyl, benzothiadiazolyl, furopyridinyl, thienopyridinyl, pyrropyridinyl, oxazolopyridinyl, thiazolopyridinyl, imidazopyridinyl and the like.

Examples of optionally substituted cycloalkyl include cyclopropyl, cyclobutyl, cyclopentyl, cyclohexyl, cycloheptyl, adamantyl and the like.

Examples of optionally substituted heterocycloalkyl include piperidyl, 4-morpholyl, 4-piperazinyl, pyrrolidinyl, perhydropyrrolidinyl, 1,4-diazaperhydroepinyl, 1,3-dioxanyl, 1,4-dioxanyl, and the like.

Examples of the optionally substituted arylalkyl group include unsubstituted arylalkyl or arylalkyl having an alkyl group such as benzyl, a-methylbenzyl, phenethyl, α-methylphenethyl, α,α-dimethylbenzyl, α,α-dimethylphenethyl, 4-methylphenethyl, 4-methylbenzyl, 4-isopropylbenzyl and the like; arylalkyl having an aryl group or an arylalkyl group such as 4-benzylbenzyl, 4-phenethylbenzyl, 4-phenylbenzyl and the like; arylalkyl having a substituted oxy group such as 4-methoxybenzyl, 4-n-tetradecyloxybenzyl, 4-n-heptadecyloxybenzyl, 3,4-dimethoxybenzyl, 4-methoxymethylbenzyl, 4-vinyloxymethylbenzyl, 4-benzyloxybenzyl, 4-phenethyloxybenzyl and the like; arylalkyl having a hydroxyl group such as 4-hydroxybenzyl, 4-hydroxy-3-methoxybenzyl, hydroxyphenethyl and the like; arylalkyl having a halogen atom such as 4-fluorobenzyl, 3-chlorobenzyl, 3,4-dichlorobenzyl and the like; 2-furfuryl, diphenylmethyl, 1-naphthylmethyl, 2-naphthylmethyl and the like.

Examples of the optionally substituted heteroarylalkyl group include 2-pyridylmethyl, 3-pyridylmethyl, 2-pyrimidinylmethyl, 5-pyrimidinylmethyl, 3-pyridazinylmethyl, 2-indolylmethyl, 5-indolylmethyl, 2-benzofuranylmethyl, 5-indolylmethyl, 2-benzothienylmethyl, 5-benzothienylmethyl, 6-fluoro-2-benzofuranylmethyl, 6-chloro-2-benzofuranylmethyl, 6-methoxy-2-benzofuranylmethyl, 6-fluoro-2-benzothienylmethyl, 6-chloro-2-benzothienylmethyl, 6-methoxy-2-benzothienylmethyl and 6-phenyl-3-pyridazinylmethyl and the like.

Examples of the optionally substituted cycloalkylalkyl group include cyclopropylmethyl, fluorocyclopropylmethyl, chlorocyclopropylmethyl, bromocyclopropylmethyl, iodocyclopropylmethyl, methylcyclopropylmethyl, 1,1-dimethylcyclopropylmethyl, 1,2-dimethylcyclopropylmethyl, hydroxycyclopropylmethyl, methoxycyclopropylmethyl, ethoxycyclopropylmethyl, methoxycarbonylcyclopropylmethyl, methylcarbamoylcyclopropylmethyl, cyclopropylethyl, cyclohexylmethyl, cyclopropylhexyl and the like.

Examples of the optionally substituted heterocycloalkylalkyl group include (2-tetrahydrofuryl) methyl, (2-tetrahydrothiofuranyl) methyl and the like.

In another embodiment of the formula (I), R₂ is optionally substituted alkyl (e.g., isobutyl, aminocarbonylethyl), or optionally substituted arylalkyl (e.g., benzyl, hydroxybenzyl).

In one embodiment of the formula (I), R₃ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl, aminocarbonylmethyl, aminocarbonylethyl, aminocarbonylpropyl and the like.

Examples of optionally substituted alkenyl group including ethenyl, allyl, 1-propenyl, 2-methylallyl and the like.

Examples of optionally substituted alkynyl group include ethynyl, 1-propynyl, and the like.

Examples of optionally substituted aryl and optionally substituted heteroaryl include biphenyl, phenyl, pyridyl, pyrimidyl, pyridazinyl, pyrazinyl, triazinyl, pyrrolyl, thienyl, furyl, thiazolyl, oxazolyl, imidazolyl, tetrahydronaphthyl, naphthyl, quinolinyl, isoquinolinyl, quinazolinyl, quinoxalinyl, cinnolinyl, naphthyridinyl, benzotriazinyl, indenyl, pyridopyrimidinyl, pyridopyrazinyl, pyridopyridazinyl, pyridotriazinyl, benzofuryl, benzothienyl, indolyl, indazolyl, benzoxazolyl, benzimidazolyl, benzothiazolyl, benzothiadiazolyl, furopyridinyl, thienopyridinyl, pyrropyridinyl, oxazolopyridinyl, thiazolopyridinyl, imidazopyridinyl and the like.

Examples of optionally substituted cycloalkyl include cyclopropyl, cyclobutyl, cyclopentyl, cyclohexyl, cycloheptyl, adamantyl and the like.

Examples of optionally substituted heterocycloalkyl include piperidyl, 4-morpholyl, 4-piperazinyl, pyrrolidinyl, perhydropyrrolidinyl, 1,4-diazaperhydroepinyl, 1,3-dioxanyl, 1,4-dioxanyl, and the like.

Examples of the optionally substituted arylalkyl group include unsubstituted arylalkyl or arylalkyl having an alkyl group such as benzyl, α-methylbenzyl, phenethyl, α-methylphenethyl, α,α-dimethylbenzyl, α,α-dimethylphenethyl, 4-methylphenethyl, 4-methylbenzyl, 4-isopropylbenzyl and the like; arylalkyl having an aryl group or an arylalkyl group such as 4-benzylbenzyl, 4-phenethylbenzyl, 4-phenylbenzyl and the like; arylalkyl having a substituted oxy group such as 4-methoxybenzyl, 4-n-tetradecyloxybenzyl, 4-n-heptadecyloxybenzyl, 3,4-dimethoxybenzyl, 4-methoxymethylbenzyl, 4-vinyloxymethylbenzyl, 4-benzyloxybenzyl, 4-phenethyloxybenzyl and the like; arylalkyl having a hydroxyl group such as 4-hydroxybenzyl, 4-hydroxy-3-methoxybenzyl, hydroxyphenethyl and the like; arylalkyl having a halogen atom such as 4-fluorobenzyl, 3-chlorobenzyl, 3,4-dichlorobenzyl and the like; 2-furfuryl, diphenylmethyl, 1-naphthylmethyl, 2-naphthylmethyl and the like.

Examples of the optionally substituted heteroarylalkyl group include 2-pyridylmethyl, 3-pyridylmethyl, 2-pyrimidinylmethyl, 5-pyrimidinylmethyl, 3-pyridazinylmethyl, 2-indolylmethyl, 5-indolylmethyl, 2-benzofuranylmethyl, 5-indolylmethyl, 2-benzothienylmethyl, 5-benzothienylmethyl, 6-fluoro-2-benzofuranylmethyl, 6-chloro-2-benzofuranylmethyl, 6-methoxy-2-benzofuranylmethyl, 6-fluoro-2-benzothienylmethyl, 6-chloro-2-benzothienylmethyl, 6-methoxy-2-benzothienylmethyl and 6-phenyl-3-pyridazinylmethyl and the like.

Examples of the optionally substituted cycloalkylalkyl group include cyclopropylmethyl, fluorocyclopropylmethyl, chlorocyclopropylmethyl, bromocyclopropylmethyl, iodocyclopropylmethyl, methylcyclopropylmethyl, 1,1-dimethylcyclopropylmethyl, 1,2-dimethylcyclopropylmethyl, hydroxycyclopropylmethyl, methoxycyclopropylmethyl, ethoxycyclopropylmethyl, methoxycarbonylcyclopropylmethyl, methylcarbamoylcyclopropylmethyl, cyclopropylethyl, cyclohexylmethyl, cyclopropylhexyl and the like.

Examples of the optionally substituted heterocycloalkylalkyl group include (2-tetrahydrofuryl) methyl, (2-tetrahydrothiofuranyl) methyl and the like.

In another embodiment of the formula (I), R₃ is hydrogen, optionally substituted alkyl (e.g., isobutyl, hydroxymethyl, methylthioethyl, aminocarbonylethyl), or optionally substituted arylalkyl (e.g., benzyl, hydroxybenzyl, hydroxyphenethyl).

In one embodiment of the formula (I), R₄ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl, aminocarbonylmethyl, aminocarbonylethyl, aminocarbonylpropyl and the like.

Examples of optionally substituted alkenyl group including ethenyl, allyl, 1-propenyl, 2-methylallyl and the like.

Examples of optionally substituted alkynyl group include ethynyl, 1-propynyl, and the like.

Examples of optionally substituted aryl and optionally substituted heteroaryl include biphenyl, phenyl, pyridyl, pyrimidyl, pyridazinyl, pyrazinyl, triazinyl, pyrrolyl, thienyl, furyl, thiazolyl, oxazolyl, imidazolyl, tetrahydronaphthyl, naphthyl, quinolinyl, isoquinolinyl, quinazolinyl, quinoxalinyl, cinnolinyl, naphthyridinyl, benzotriazinyl, indenyl, pyridopyrimidinyl, pyridopyrazinyl, pyridopyridazinyl, pyridotriazinyl, benzofuryl, benzothienyl, indolyl, indazolyl, benzoxazolyl, benzimidazolyl, benzothiazolyl, benzothiadiazolyl, furopyridinyl, thienopyridinyl, pyrropyridinyl, oxazolopyridinyl, thiazolopyridinyl, imidazopyridinyl and the like.

Examples of optionally substituted cycloalkyl include cyclopropyl, cyclobutyl, cyclopentyl, cyclohexyl, cycloheptyl, adamantyl and the like.

Examples of optionally substituted heterocycloalkyl include piperidyl, 4-morpholyl, 4-piperazinyl, pyrrolidinyl, perhydropyrrolidinyl, 1,4-diazaperhydroepinyl, 1,3-dioxanyl, 1,4-dioxanyl, and the like.

Examples of the optionally substituted arylalkyl group include unsubstituted arylalkyl or arylalkyl having an alkyl group such as benzyl, α-methylbenzyl, phenethyl, α-methylphenethyl, α,α-dimethylbenzyl, α,α-dimethylphenethyl, 4-methylphenethyl, 4-methylbenzyl, 4-isopropylbenzyl and the like; arylalkyl having an aryl group or an arylalkyl group such as 4-benzylbenzyl, 4-phenethylbenzyl, 4-phenylbenzyl and the like; arylalkyl having a substituted oxy group such as 4-methoxybenzyl, 4-n-tetradecyloxybenzyl, 4-n-heptadecyloxybenzyl, 3,4-dimethoxybenzyl, 4-methoxymethylbenzyl, 4-vinyloxymethylbenzyl, 4-benzyloxybenzyl, 4-phenethyloxybenzyl and the like; arylalkyl having a hydroxyl group such as 4-hydroxybenzyl, 4-hydroxy-3-methoxybenzyl, 4-hydroxyphenethyl and the like; arylalkyl having a halogen atom such as 4-fluorobenzyl, 3-chlorobenzyl, 3,4-dichlorobenzyl and the like; 2-furfuryl, diphenylmethyl, 1-naphthylmethyl, 2-naphthylmethyl and the like.

Examples of the optionally substituted heteroarylalkyl group include 2-pyridylmethyl, 3-pyridylmethyl, 2-pyrimidinylmethyl, 5-pyrimidinylmethyl, 3-pyridazinylmethyl, 2-indolylmethyl, 5-indolylmethyl, 2-benzofuranylmethyl, 5-indolylmethyl, 2-benzothienylmethyl, 5-benzothienylmethyl, 6-fluoro-2-benzofuranylmethyl, 6-chloro-2-benzofuranylmethyl, 6-methoxy-2-benzofuranylmethyl, 6-fluoro-2-benzothienylmethyl, 6-chloro-2-benzothienylmethyl, 6-methoxy-2-benzothienylmethyl and 6-phenyl-3-pyridazinylmethyl and the like.

Examples of the optionally substituted cycloalkylalkyl group include cyclopropylmethyl, fluorocyclopropylmethyl, chlorocyclopropylmethyl, bromocyclopropylmethyl, iodocyclopropylmethyl, methylcyclopropylmethyl, 1,1-dimethylcyclopropylmethyl, 1,2-dimethylcyclopropylmethyl, hydroxycyclopropylmethyl, methoxycyclopropylmethyl, ethoxycyclopropylmethyl, methoxycarbonylcyclopropylmethyl, methylcarbamoylcyclopropylmethyl, cyclopropylethyl, cyclohexylmethyl, cyclopropylhexyl and the like.

Examples of the optionally substituted heterocycloalkylalkyl group include (2-tetrahydrofuryl) methyl, (2-tetrahydrothiofuranyl) methyl and the like.

In another embodiment of the formula (I), R₄ is hydrogen, optionally substituted alkyl (e.g., methyl, isopentyl), optionally substituted aryl (e.g., phenyl), or optionally substituted arylalkyl (e.g., phenethyl, hydroxyphenethyl).

In another embodiment of the formula (I), Q is —CH₂—.

In another embodiment of the formula (I), Q is —CH₂CH₂—

In another embodiment of the formula (I), V is a bond, —CO—, —SO₂—, —NHCO—, or —OCO—.

In another embodiment of the formula (I), V is a bond, or —CO—.

A preferred embodiment of the formula (I) is a compound having the following formula (II) or a pharmaceutically acceptable salt thereof:

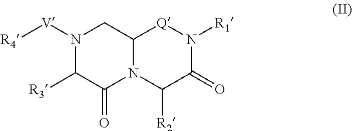

wherein
R₁′ is hydrogen, optionally substituted alkyl, optionally substituted heterocycloalkyl, or optionally substituted arylalkyl;
R₂′ is optionally substituted alkyl, or optionally substituted arylalkyl;
R₃′ is hydrogen, optionally substituted alkyl, or optionally substituted arylalkyl;
Q′ is —CH₂— or —CH₂CH₂—;
V′ is a bond, or —CO—; and
R₄′ is hydrogen, optionally substituted alkyl, optionally substituted aryl, or optionally substituted arylalkyl.

In the following, a compound having the formula (I) or the formula (II) is to be also referred to as “the compound of the present invention”.

The general synthesis of the compound of the present invention is described in the following “Production Method”. Abbreviations used in the Production Method and Examples are as follows.
AcOH: acetic acid
AC₂O: acetic anhydride
aq.: aqueous solution
AZADOL: 2-Hydroxy-2-azaadamantane
Cbz: benzyloxycarbonyl
Bn: benzyl
Boc: tert-butoxycarbonyl
Boc₂O: di-tert-butyl dicarbonate
CHCl₃: chloroform
DCM: dichloromethane
DIEA: N,N-diisopropylethylamine
DIPEA: N-ethyl-N-isopropyl-propan-2-amine
DMB: 2,4-dimethoxybenzyl
DMF: N,N-dimethylformamide
DMSO: dimethyl sulfoxide
EDCl: 1-(3-dimethylaminopropyl)-3-ethylcarbodiimide
EtO (OEt): ethoxy
EtOAc: ethyl acetate
Fmoc: 9-fluorenylmethyloxycarbonyl
HATU: 1-[bis(dimethylamino)methylene]-1H-1,2,3-triazolo[4,5-b]pyridinium 3-oxide hexafluorophosphate
HOBt: 1-hydroxybenzotriazole
hr: hour (s)
MeCN: acetonitrile
MeO (OMe): methoxy
MeOH: methanol
MsO: methanesulfonyloxy
OAc (AcO) : acetoxy
PE/EA: petroleum ether/ethyl acetate
Ph: phenyl
rt: room temperature
sat.: saturated
TBAF: tetrabutylammonium Fluoride
TBS: tert-butyldimethylsilyl
TBDPS: tert-butyldiphenylsilyl
tBu: tert-butyl
TEA: triethylamine
TFA: trifluoroacetic acid
THF: tetrahydrofuran
TMSOTf: trimethylsilyl triflate
Trt: trityl
TsO: toluenesulfonyloxy
Production Method

In the formula, R₁-R₄, V and Q are as defined in the aforementioned compound (I).

In the formula, U is —COCl, —CO₂H, —CHO, —SO₂Cl, —NCO, or —OCOCl.

While Boc group is recited as a nitrogen atom protecting group, TBDMS group is recited as a hydroxyl protecting group, and Cl group and Br group are recited as leaving groups in the formula, these are not limitative and generally-known nitrogen atom protecting groups (e.g., Cbz group, Bn group, etc.), hydroxyl protecting groups (e.g., Bn group etc.), and leaving groups (e.g., MsO group, TsO group, etc.) may also be used.

Each compound in the formula may form a salt as necessary.

Each step is explained in the following. The reaction conditions in each step are not limited to those described below, and generally-known reagents and reaction conditions are applicable.

### Production of Compound (I)

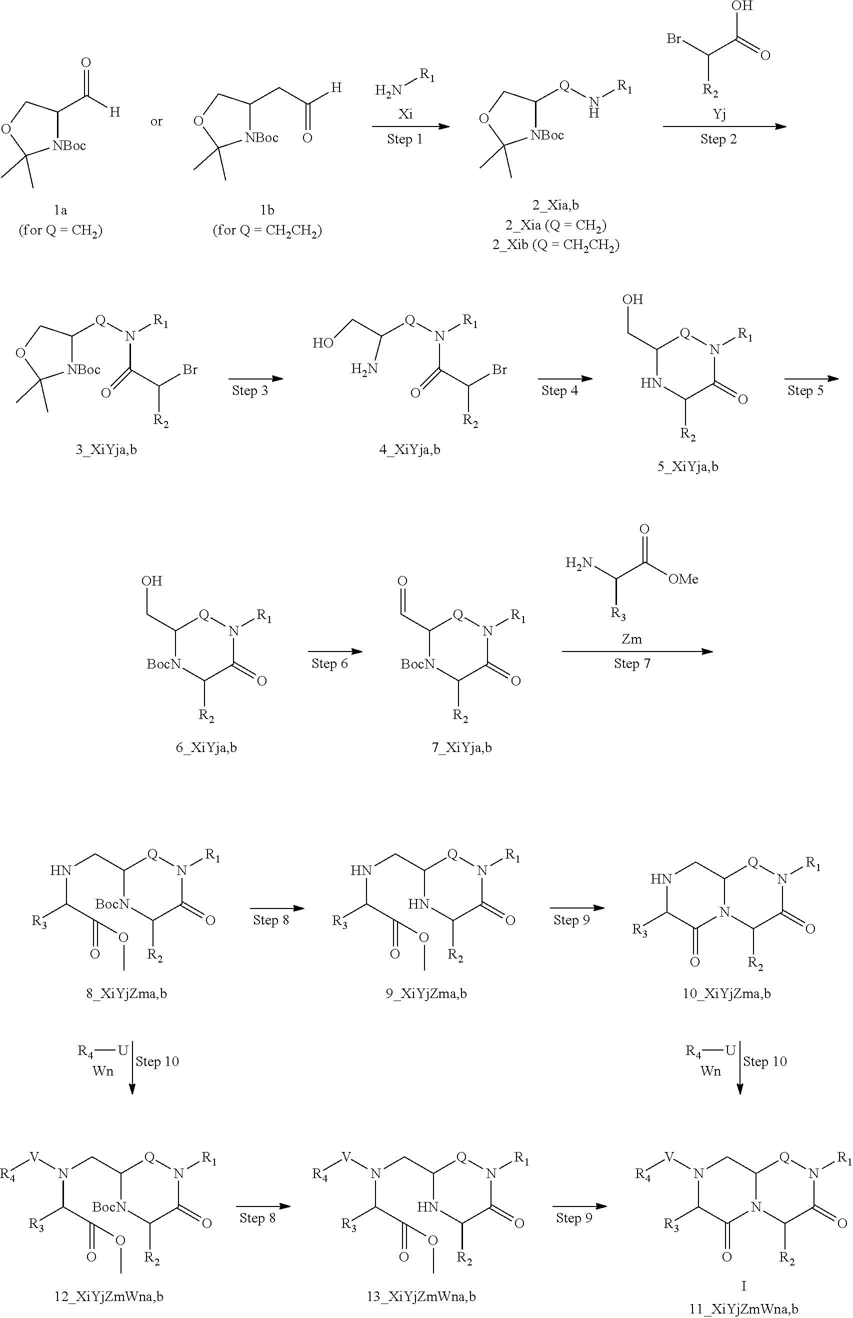

The compound number of the derivative of compound (I) is compound 11_XiYjZmWna,b.

When R₄ is hydrogen and V is a bond in the formula of compound 11_XiYjZmWna,b, compound 11_XiYjZmWna,b is compound 10_XiYjZma,b.

### [Step 1] Reductive Alkylation

By reacting a known compound: reagent 1a (Dondoni, Alessandro, et al., Organic Syntheses (2000), 77, 64-77.), or 1b (Ouathek Ouerfelli, et al., Synlett (1993), 6, 409-410.) with reagent Xi and a reducing agent in a solvent, the corresponding compounds 2_Xia and 2_Xib (compound 2_Xia,b) can be synthesized. NaBH(OAc)₃ or NaBH₃CN is preferable as the reducing agent, and DCM or MeOH is preferable as the solvent. The reaction temperature is preferably 0-20° C. and the reaction time is preferably 16 hr.

### [Step 2] Amidation

By reacting 2_Xia,b with Yj, a condensing agent, and a base in a solvent, compound 3_XiYja,b (compounds 3_XiYja (Q═CH₂), 3_XiYjb (Q═CH₂CH₂)) can be synthesized. EDCI-HOBt or HATU is preferable as the condensing agent, and 2,4,6-trimethylpyridine or DIPEA is preferable as the base. As the solvent, DCM is preferable. The reaction temperature is preferably 0-20° C. and the reaction time is preferably 16 hr.

### [Step 3] Deprotection

By reacting 3_XiYja,b with a deprotecting agent in a solvent, compound 4_XiYja,b (compounds 4_XiYja (Q═CH₂), 4_XiYjb (Q═CH₂CH₂)) can be synthesized. TFA, TMSOTf-TEA, or H₂SO₄ is preferable as the deprotecting agent. As the solvent, DCM, water, or a mixed solvent thereof is preferable. The reaction temperature is preferably 0-25° C. and the reaction time is preferably 1 to 3 hr.

### [Step 4] Ring Closure

By reacting 4_XiYja,b with a base in a solvent, compound 5_XiYja,b (compounds 5_XiYja (Q═CH₂), 5_XiYjb (Q═CH₂CH₂)) can be synthesized. Na₂CO₃ is preferable as the base, and MeCN, water, or a mixed solvent thereof is preferable as the solvent. The reaction temperature is preferably −30 to 20° C. and the reaction time is preferably 1 to 16 hr.

### [Step 5] Protection

By reacting 5_XiYja,b with a protecting agent in a solvent, compound 6_XiYja,b (compounds 6_XiYja (Q═CH₂), 6_XiYjb (Q═CH₂CH₂)) can be synthesized. Boc₂O is preferable as the protecting agent. A base may be added and DIPEA is preferable as the base. As the solvent, DCM is preferable. The reaction temperature is preferably 20° C. and the reaction time is preferably 24 hr.

### [Step 6] Hydroxyl Group Oxidation

By reacting 6_XiYja,b with an oxidant in a solvent, compound 7_XiYja,b (compounds 7_XiYja (Q═CH₂) , 7_XiYjb (Q═CH₂CH₂)) can be synthesized. As the oxidant, DMSO-(COCl)₂ is preferable. A base may be added and DIPEA is preferable as the base. As the solvent, DCM is preferable. The reaction temperature is preferably −78 to 0° C. and the reaction time is preferably 2 hr.

As other oxidant, NaClO can also be used. In this case, AZADOL is added as the catalyst, KBr is added as the additive, and NaHCO₃ is added as the base. As the solvent, a mixed solvent of DCM and water is preferable. The reaction temperature is preferably 0° C. and the reaction time is preferably 0.5 hr.

### [Step 7] Reductive Alkylation

By performing [step 1] using 7_XiYja,b and Zm, compound 8_XiYjZma,b (compounds 8_XiYjZma (Q═CH₂) , 8_XiYjZmb (Q═CH₂CH₂)) can be synthesized.

### [Step 8] Deprotection

By performing [step 3] using 8_XiYjZma,b, compound 9_XiYjZma,b (compounds 9_XiYjZma (Q═CH₂), 9_XiYjZmb (Q═CH₂CH₂)) can be synthesized.

### [Step 9] Ring Closure

By heating 9_XiYjZma,b with an additive in a solvent, compound 10_XiYjZma,b (compounds 10_XiYjZma (Q═CH₂) , 10_XiYjZmb (Q═CH₂CH₂)) can be synthesized. AcOH is preferable as the additive, and MeCN or toluene is preferable as the solvent. The reaction temperature is preferably 50-80° C. and the reaction time is preferably 4 to 12 hr.

Alternatively, 10_XiYjZma,b can be synthesized by converting the ester group of 9_XiYjZma,b to carboxylic acid and then performing [Step 2]. In the formula, the ester group is a methyl ester group, though it is not limited to the methyl ester group. The ester group can be converted to a carboxylic acid by, for example, deprotection with an acid when the ester group is t-butyl ester. In this case, the conversion can also be performed simultaneously with the deprotection in [Step 8].

### [Step 10] Acylation/Alkylation

By reacting 10_XiYjZma,b with various R₄—U (Wn) or an acid anhydride ((R₄—CO)₂O) in a solvent, compound 11_XiYjZmWna,b (compounds 11_XiYjZmWna (Q═CH₂), 11_XiYjZmWnb (Q═CH₂CH₂)) can be synthesized.

For example, by reacting R₄—U (Wn: U═COCl) or (R₄—CO)₂O and a base in a solvent, or by performing [step 1] using R₄—U (Wn: U═CHO) , or by performing [step 2] using R₄—U (Wn: U═CO₂H), compound 11_XiYjZmWna,b can be synthesized.

In the case of R₄—U (Wn: U═COCl) or (R₄—CO)₂O, TEA is preferable as the base, and DCM is preferable as the solvent. The reaction temperature is preferably 0-20° C. and the reaction time is preferably 16 hr.

Compound 11_XiYjZmWna,b can also be synthesized by subjecting 8_XiYjZma,b to [step 10], [step 8], and [step 9] in this order.

Commercially available products of the aforementioned Xi, Yj, Zm, and Wn can be used or they can be synthesized by known synthesis methods.

By using optically active forms of 1 (1a or 1b), Yj, and Zm, the steric structures at the 3, 6, and 9a-positions of compound 11_XiYjZmWna,b can be controlled.

In this case, compound (I) having any steric chemistry can be produced by isolating the diastereomer in any step of [step 1]-[step 10] of the aforementioned production method.

For example, the diastereomer can be isolated by purifying 6_XiYja,b, or by protecting a hydroxyl group, followed by purification. As a purification method, silica gel column chromatography and preparative HPLC are preferable.

An example is shown below.

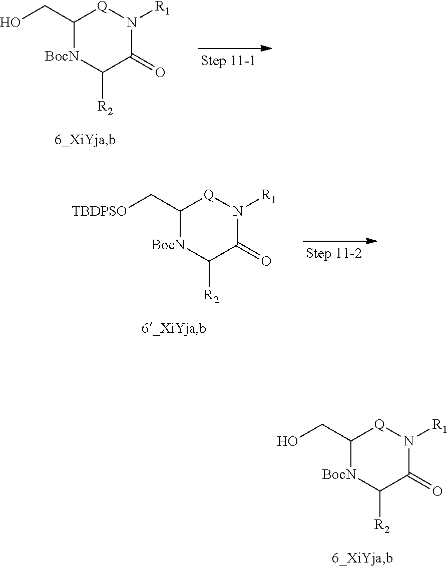

### [Step 11-1] Protection of Hydroxyl Group

By reacting 6_XiYja,b with a silylating agent and a base in a solvent, compound 6′_XiYja,b can be synthesized. TBDPSCl is preferable as the silylating agent, and imidazole is preferable as the base. As the solvent, dichloromethane is preferable. The reaction temperature is preferably 20° C. and the reaction time is preferably 16 hr. By protecting the hydroxyl group in this way, the diastereomer can be isolated by silica gel column chromatography or preparative HPLC.

### [Step 11-2] Deprotection of Hydroxyl Group

By reacting the thus-obtained 6′_XiYja,b with a deprotecting agent in a solvent, compound 6_XiYja,b can be synthesized. TBAF is preferable as the deprotecting agent, and THF is preferable as the solvent. The reaction temperature is preferably 20° C. and the reaction time is preferably 0.5 hr.

In the aforementioned production method, any of the substituents R₁-R₄ can be converted to a different substituent by a functional group conversion reaction at any position in the formula.

When a protecting group is necessary for any of R₁-R₄ in the formula, protection and deprotection can be performed at any position in the formula.

An example is shown below.

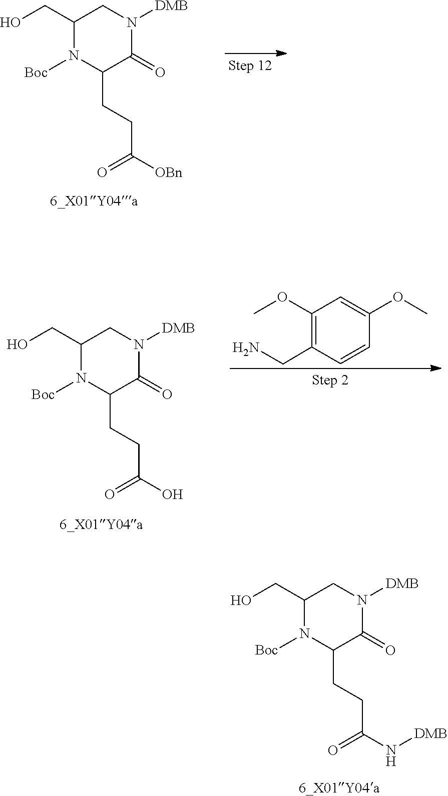

### [Step 12] Deprotection

By deprotecting 6X01″Y04″″ a by catalytic hydrogenation, 6_X01′'Y04″ a can be synthesized. Pd/C is preferable as the catalyst, and the reaction is preferably performed under a hydrogen atmosphere as the hydrogen source. As the solvent, MeOH is preferable. The reaction temperature is preferably 0-20° C. and the reaction time is preferably 2 hr.

By subjecting the obtained 6_X01″Y04″a to [step 2] using 2,4-dimethoxybenzylamine, compound 6_X01″Y04′a can be synthesized.

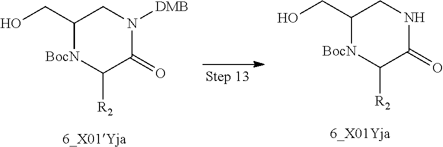

### [Step 13] Deprotection of Amide Group

By reacting 6_X01′Yja with a deprotecting agent in a solvent, compound 6_X01Yja can be synthesized. Li—NH₃ (liq.) is preferable as the deprotecting agent. As the solvent, THF is preferable. The reaction temperature is preferably −78° C. and the reaction time is preferably 2 hr.

Furthermore continuing the explanation, the compounds of the present invention, salts thereof and derivatives thereof are excellent in selectivity pharmacological action, safety (various toxicities and safety pharmacology), pharmacokinetic performance, physicochemical property and the like, and therefore the usefulness as active ingredients of medicaments can be confirmed.

Examples of tests concerning pharmacological action selectivity include, but not be limited to, inhibition or activation assays on various pharmacological target receptors, inhibition assays on various pharmacological target enzymes, ion channels or transporters, cell tests to be used for the evaluation for various pharmacological action, and the like.

Examples of tests concerning safety include, but not be limited to, the following list including cytotoxic tests (e.g., tests using HL60 cells, hepatocytes, etc., and the like), genotoxicity tests (e.g., Ames test, mouse lymphoma TK test, chromosomal aberration test, micronucleus test and the like), skin sensitization tests (e.g., Buehler method, GPMT method, APT method, LLNA test and the like), skin photosensitization tests (e.g., Adjuvant and Strip method and the like), eye irritation tests (e.g., single instillation, short-term continuation instillation, repetitive instillation and the like), safety pharmacology tests for the cardiovascular system (e.g., telemetry method, APD method, hERG inhibition assay and the like), safety pharmacology tests for the central nervous system (e.g., FOB method, modified version of Irwin method and the like), safety pharmacology tests for the respiratory system (e.g., measurement method using a respiratory function measuring apparatus, measurement method using a blood gas analyzer and the like), general toxicity tests, and the like.

Examples of tests concerning pharmacokinetic performance include, but not be limited to, the following list including cytochrome P450 enzyme inhibition or induction tests, cell permeability tests (e.g., tests using CaCO-2 cells, MDCK cells etc., and the like), drug transporter ATPase assay, oral absorption tests, blood concentration transition measurement tests, metabolism tests (e.g., stability test, metabolite molecular species test, reactivity test and the like), solubility tests (e.g., solubility test based on turbidity method and the like), and the like.

Examples of tests concerning physicochemical property include, but not be limited to, the following list including chemical stability test (e.g., stability test using HPLC etc., and the like), partition coefficient (e.g., partition test using octanol phase/water phase and the like), ionization constant test, crystallization test, and the like.

In another embodiment, a method for treating various diseases by administering the compound of the present invention is provided. The compound of the present invention may be used for preventing or treating diseases controlled by Notch signal transduction pathway.

In one embodiment, screening relating to the inhibitory action of the Notch signal transduction pathway is performed using a doxycycline-inducing lentiviral vector (see Examples for specific procedures).

The test compound here is a compound described in the present specification, that is, the compound of the present invention. Typically, test compounds are tested at several different concentrations, and the concentrations are partly selected according to the assay conditions.

The compound of the present invention may inhibit Notch signal transduction by interacting with the Notch intracellular domain.

The present invention is also related to prodrugs using the libraries containing one or more compounds of the present invention. A prodrug is typically designed to release the active drug in the body during or after absorption by enzymatic and/or chemical hydrolysis. The prodrug approach is an effective means of improving the oral bioavailability or i.v. administration of poorly water-soluble drugs by chemical derivatization to more water-soluble compounds. The most commonly used prodrug approach for increasing aqueous solubility of drugs containing a hydroxyl group is to produce esters containing an ionizable group; e.g., phosphate group, carboxylate group, alkylamino group (Fleisher et al., Advanced Drug Delivery Reviews, 115-130, 1996; Davis et al., Cancer Res., 7247-7253).

In other aspects, the present invention provides pharmaceutical compositions containing a compound of the present invention. These compositions may be used in various methods of the present invention as described in detail below.

The pharmaceutical composition of the present invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions (e.g., injection) used for parenteral (particularly, intravenous) , intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In addition, pH may be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound, e.g., a compound having general formula (I) in the required amount, in an appropriate solvent with one or a combination of ingredients enumerated above, as required, followed by filtered sterilization. Generally, dispersions are prepared by incorporating the active compound into a sterile vehicle that contains a dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules.

Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent I such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It can be advantageous to formulate oral or parenteral compositions in dosage unit form for ease of administration and uniformity of dosage. Dosage unit form as used herein refers to physically discrete units suited as unitary dosages for the subject to be treated; each unit containing a 5 predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For instance, in certain embodiments, a pharmaceutical composition of the present invention is one suitable for oral administration in unit dosage form such as a tablet or capsule that contains from about 1 mg to about 1 g of the compound of this invention. In some other embodiments, a pharmaceutical composition of the present invention is one suitable for intravenous, subcutaneous or intramuscular injection. A patient may receive, for example, an intravenous, subcutaneous or intramuscular dose of about 1 μg/kg to about 1 g/kg of the compound of the present invention. The intravenous, subcutaneous and intramuscular dose may be given by means of a bolus injection or by continuous infusion over a period of time. Alternatively a patient will receive a daily oral dose approximately equivalent to the daily parenteral dose, the composition being administered 1 to 4 times per day.

Preferably, the compound of the formula (I) of the present invention can be administered intravenously (particularly preferably, by continuous drip infusion or rapid intravenous administration) to mammals inclusive of human.

In the case, the dose is selected appropriately depending on various factors such as the body weight and/or age of patients, and/or the degree of the symptom and an administration route. For example, the dose of the compound of the formula (I) for intravenous administration is generally in the range of 1 to 10000 mg/day/m² human body surface area, preferably in the range of 1 to 5000 mg/day/m² human body surface area, and more preferably 10 to 5000 mg/day/m² human body surface area by continuous drip infusion administration.

A pharmaceutical composition containing the compound of the present invention can be used for diseases regulated by Notch signal transduction pathway. More specifically, a compound that inhibits Notch signal provides a method for suppressing expression of Hes1 and Hes5 and promoting differentiation of neural stem cells, and is expected to be a candidate for a new nerve regeneration drug.

The present invention also provides methods for promoting differentiation of a neural stem cell comprising contacting a neural stem cell with a compound according to formula (I) in an amount effective to promote differentiation of a neural stem cell. Such methods are also useful in treating neurodegenerative diseases (e.g., glaucoma, macular degeneration, Parkinson's Disease, and Alzheimer's disease) and injuries to nervous system. “Neural stem cell” refers to a clonogenic, undifferentiated, multipotent cell capable of differentiating into a neuron, an astrocyte or an oligodendrocyte under appropriate conditions. A compound promotes differentiation of neural stem cells if neural stem cells exhibit a statistically significantly higher degree of differentiation in the presence of the compound than in the absence of the compound. Such a compound may be identified using assays involving in vitro cultured stem cells or animal models (Albranches et al., Biotechnol. Lett. 25: 725-30, 2003; Deng et al., Exp. Neurol. 182: 373-82, 2003; Munoz-Elias et al., Stem Cells 21: 437-48, 2003; Kudo et al, Biochem. Pharmacol. 66: 289-95, 2003; Wan et al., Chin. Med. J. 116: 428-31, 2003; Kawamorita et al., Hum. Cell 15: 178-82, 2002; Stavridis and Smith, Biochem. Soc. Trans. 31: 45-9, 2003; Pachemik et al., Reprod. Nutr. Dev. 42: 317-26, 2002; Fukunaga et al., supra). The neural stem cell may be a cultured stem cell, a stem cell freshly isolated from its source tissue, or a stem cell within its source organism. Thus, contacting the neural stem cell with a compound according to the present invention may be carried out either in vitro (for a cultured or freshly isolated stem cell) or in vivo (for a stem cell within its source organism). The resulting differentiated neural cell, if generated in vitro, may be transplanted into a tissue in need thereof (Lacza et al., supra; Chu et al., supra; Fukunaga et al., supra). Such a tissue includes a brain tissue or other nervous tissue that suffers from a trauma or a neurodegenerative disease.

The following non-limiting examples illustrate the compounds, compositions, and methods of use of this invention.

### Examples

The present invention is explained in more detail in the following by referring to Production Examples, Examples, Reference Examples and Experimental Examples; however, the scope of the present invention is not limited thereto.

In the Examples, ¹H NMR was measured using Bruker AVANCE III 400; Bruker AVANCE III 400 HD & Bruker AVANCE NEO 40. LC/MS was measured using Agilent 1100 LC & Agilent G1956A, (ELSD: 1260 Infinity) or using Shimazu UFLC/MS System (Shimazu-2020 mass spectrometer, ODS column for the chromatography column). LC/MS analysis with Agilent system was performed under the conditions described in Table 1 or Table 2. LC/MS analysis with Shimazu UFLC/MS System was performed under the conditions described below; water with 0.04% TFA and acetonitrile with 0.04% TFA were used as a mobile phase or 5 mM AcONH₄ in water and 5 mM ACONH₄ in acetonitrile were used as a mobile phase.

TABLE 1

| Instrument | | Agilent 1100 LC & Agilent G1956A | | |
|---|---|---|---|---|
| Software | | Agilent Chemstation Rev. B. 04.03[16] | | |
| HPLC | Column | Agilent ZORBAX 5 μm SB-Aq, 2.1 * 50 mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 1 | 0.8 |
| | | 0.40 | 1 | 0.8 |
| | | 3.40 | 90 | 0.8 |
| | | 3.90 | 100 | 0.8 |
| | | 3.91 | 1 | 0.8 |
| | | 4.00 | 1 | 1.0 |
| | | 4.50 | 1 | 1.0 |

| | | |
|---|---|---|
| | Post time(min) | 0 |
| | Column Temp | 50° C. |
| | Detector | DAD |
| MS | Ionization source | ESI |
| | Drying Gas | N2 |
| | Drying Gas Flow | 10 (L/min) |
| | Nebulizer Pressure | 2070 (Torr) |
| | Drying Gas | 350° C. |
| | Temperature | |
| | Capillary Voltage | 2500 (V) |
| | MS Polarity | Positive |
| | MS Mode | Scan |
| | Mass Range | 100-1500 |

TABLE 2

| Instrument | | Agilent 1100 LC & Agilent G1956A | | |
|---|---|---|---|---|
| Software | | Agilent Chemstation Rev. B. 04.03[16] | | |
| HPLC | Column | Agilent ZORBAX 5 μm SB-Aq, 2.1 * 50 mm | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 10 | 0.8 |
| | | 0.40 | 10 | 0.8 |
| | | 3.40 | 100 | 0.8 |
| | | 3.90 | 100 | 0.8 |
| | | 3.91 | 10 | 0.8 |
| | | 4.00 | 10 | 1.0 |
| | | 4.50 | 10 | 1.0 |

| | | |
|---|---|---|
| | Post time(min) | 0 |
| | Column Temp | 50° C. |
| | Detector | DAD |
| MS | Ionization source | ESI |
| | Drying Gas | N2 |
| | Drying Gas Flow | 10 (L/min) |
| | Nebulizer Pressure | 2070 (Torr) |
| | Drying Gas | 350° C. |
| | Temperature | |
| | Capillary Voltage | 2500 (V) |
| | MS Polarity | Positive |
| | MS Mode | Scan |
| | Mass Range | 50-1500 |

General Prep-HPLC condition (FA) :

### Column: Phenomenex Gemini 25×150 mm, 5 μm

Mobile phase A: water with 0.225% v/v Formic Acid
Mobile phase B: acetonitrile
UV detection wavelength: 220 nm
Flow rate: 25 ml/min
Gradient time table:

| | |
|---|---|
| 0 min | begin_b&B(the percentage of A is 100-B) |
| 11 min | end_b% B |
| 11.2 min | 100% B |
| 13 min | 100% B |
| 13.2 min | 5% B |

The values of A and B depend on the kind of compounds.
[Example 1] Synthesis of (3S,6S,9aR)-3,6-diisobutyl-2-methylhexahydro-4H-pyrazino[1,2-a]pyrazine-4,7(6H)-dione (11_X01Y01Z01W01a)

### Example 1-1

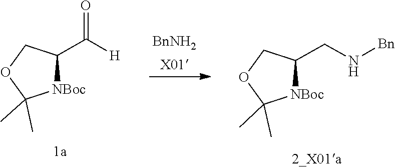

A solution of 1a (0.12 kg) and X01′ (57 g) in DCM (2.5 L) was stirred at 20° C. for 1 hr. The NaBH(OAc)₃ (0.16 kg) was added to the mixture at 0-10° C. Then the mixture was stirred at 20° C. for 16 hr. The reaction mixture was adjusted pH to 7 with sat·Na₂CO₃aq., and extracted with DCM. The combined organic layer was dried over Na₂SO₄, filtered, and concentrated under reduced pressure to give a residue. The residue was purified by column chromatography (SiO₂, PE/EA=50/1 to 0/1) to give the compound 2_X01′a (0.12 kg) as yellow oil.

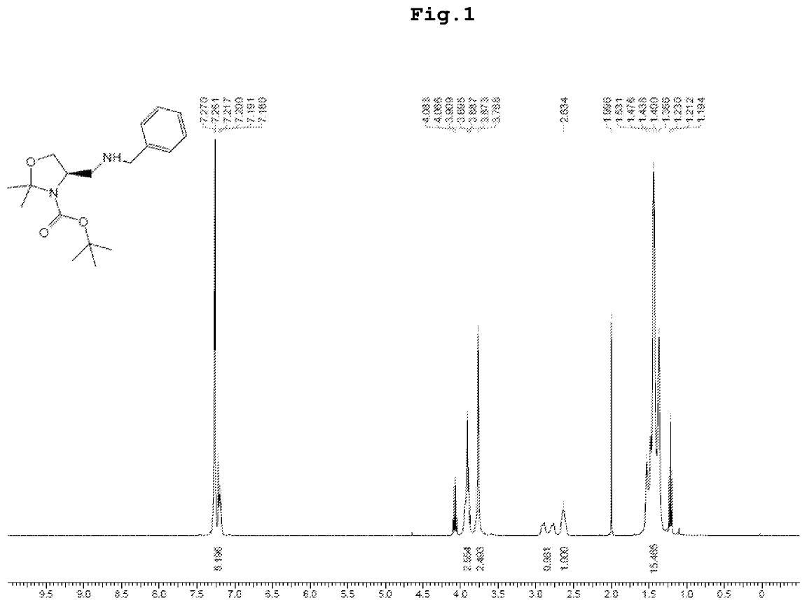

¹H NMR (400 MHZ, CDCl₃) data of 2_X01′a is shown in FIG. 1.

### Example 1-2

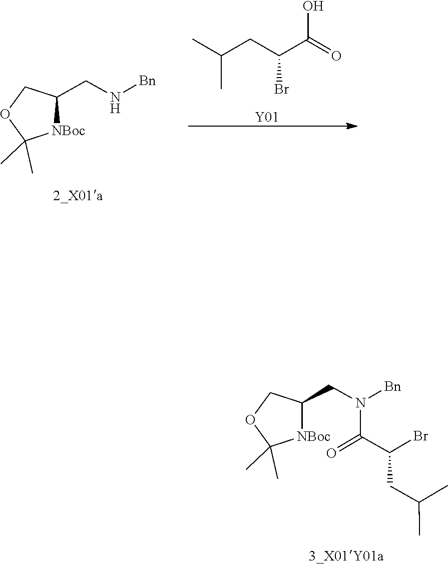

To a solution of 2_X01′a (25 g) and Y01 (21 g) in DCM (0.50 L) was added HOBt (11 g), 2,4,6-trimethy pyridine (10 g) and EDCI (16 g) at 0-10° C. The mixture was stirred at 20° C. for 16 hr. The reaction mixture was diluted with water (0.10 L). The organic layer was washed with 1 N HCl (0.10 L×2), then washed with sat·Na₂CO₃aq. (0.10 L×2), dried over Na₂SO₄, filtered, and concentrated under reduced pressure to give a residue. The residue was purified by column chromatography (SiO₂, PE/EA=100/1 to 20/1) to give 3_X01′Y01a (27 g) as yellow oil. LCMS: m/z=499.0 [M+2]⁺, 497.0[M]⁺

### Example 1-3

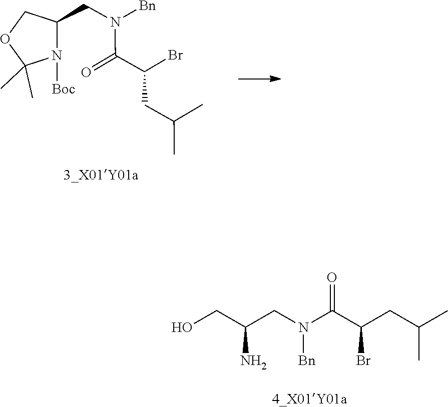 [SMILES: CC(C)C[C@@H](Br)C(=O)N(Cc1ccccc1)C[C@@H]1COC(C)(C)N1C(=O)OC(C)(C)C ; CC(C)C[C@@H](Br)C(=O)N(Cc1ccccc1)C[C@@H](N)CO]

To a solution of 3_X01′Y01a (27 g) in DCM (0.13 L) was added H₂O (4.8 mL) , then was added dropwise TFA (48 mL) at 0° C. The mixture was stirred at 20° C. for 1 hr. The reaction mixture was diluted with water (0.10 L) and extracted with EtOAc (80 mL×2). The combined organic layer was washed with 1N HCl (80 mL×2), then washed with sat·Na₂CO₃aq. (80 mL×2), dried over Na₂SO₄, filtered, and concentrated under reduced pressure to give a residue. The residue was purified by column chromatography (SiO₂, PE/EA=100/1 to 0/1) to give 4_X01′Y01a (24 g, TFA salt) as yellow oil.

LCMS: m/z=359.1 [M+2]⁺, 357.1[M]⁺

### Example 1-4

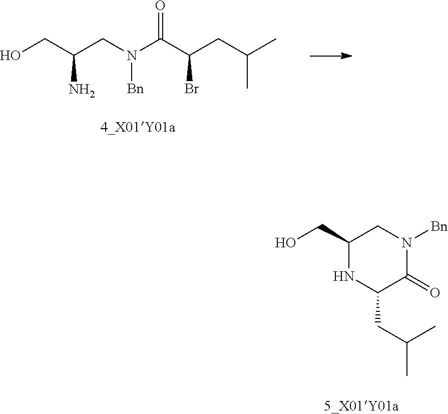 [SMILES: CC(C)C[C@@H](Br)C(=O)N(Cc1ccccc1)C[C@@H](N)CO ; CC(C)C[C@@H]1N[C@@H](CO)CN(Cc2ccccc2)C1=O]

To a solution of 4_X01′Y01a (8.0 g, TFA salt) in MeCN (85 mL) was added Na₂CO₃ (4.9 g) at −30 to −20° C. The mixture was stirred at 0° C. for 2 hr. The reaction mixture was concentrated to give a residue. The residue was diluted with water (80 mL) and extracted with EtOAc (80 mL×2). The combined organic layer was washed with brine (0.10 L×2) , dried over Na₂SO₄, filtered, and concentrated under reduced pressure to give a residue. This reaction was performed three batches. The combined residue was purified by column chromatography (SiO₂, PE/EA=10 to 0/1) to give 5_X01′Y01a (13 g) as yellow oil. LCMS: m/z=277.2 [M+1]⁺

### Example 1-5

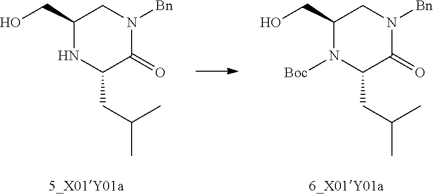 [SMILES: CC(C)C[C@@H]1N[C@@H](CO)CN(Cc2ccccc2)C1=O ; CC(C)C[C@H]1C(=O)N(Cc2ccccc2)C[C@H](CO)N1C(=O)OC(C)(C)C]

To a solution of 5_X01′Y01a (13 g) in DCM (0.30 L) was added Boc₂O (11 g) and DIEA (8.4 g). The mixture was stirred at 25° C. for 24 hr. The reaction mixture was concentrated under reduced pressure to give a residue. The residue was diluted with water (0.20 L) and extracted with EtOAc (0.15 L×2). The combined organic layers were washed with 1N HCl (0.15 L×2), then washed with sat·Na₂CO₃aq. (0.15 L×2) , brine (0.10 L×2), dried over Na₂SO₄, filtered and concentrated under reduced pressure to give a residue. The residue was purified by column chromatography (SiO₂, PE/EA=20/1 to 1/1) to give 6_X01′Y01a (12 g) as yellow oil.

LCMS: m/z=377.3 [M+1]⁺

### Example 1-6

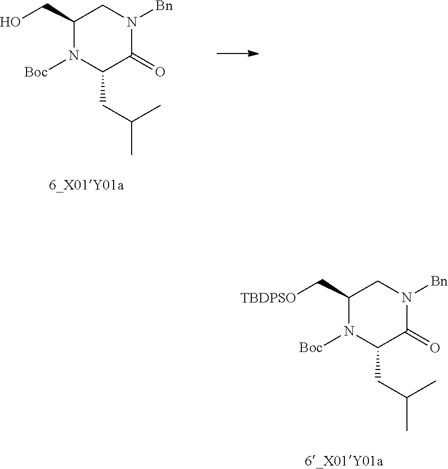

To a solution of 6_X01′Y01a (12 g) in DCM (0.14 L) was added imidazole (6.3 g) and TBDPSC1 (19 g). The mixture was stirred at 20° C. for 16 hr. The mixture was concentrated to give a residue. The residue was diluted with water (0.10 L) and extracted with EtOAc (0.10 L×2). The combined organic layers were washed with 1 N HCl (0.10 L×2), and sat·Na₂CO₃aq. (0.10 L×2), brine (0.10 L×2), dried over Na₂SO₄, filtered and concentrated under reduced pressure to give a residue. The residue was purified by column chromatography (SiO₂, PE/EA=100/1 to 20/1) to give 6′_X01′Y01a (20 g) as yellow oil.

LCMS: m/z=615.4 [M+1]⁺

### Example 1-7

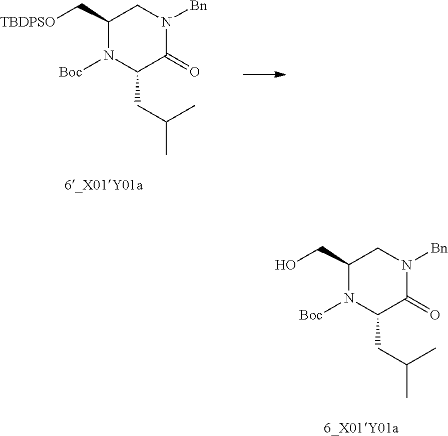

To a solution of 6′_X01′Y01a (22 g) in THE (0.25 L) was added TBAF (1 M, 72 mL) at 0° C. The mixture was stirred at 20° C. for 0.5 hr. Then the reaction mixture was diluted with water (0.20 L) and extracted with EtOAc (0.20 L×2). The combined organic layers were washed with 1N HCl (0.20 L×2) , then washed with sat·Na₂CO₃aq. (0.20 L×2), brine (0.20 L×2), dried over Na₂SO₄, filtered and concentrated under reduced pressure to give a residue. The residue was purified by column chromatography (SiO₂, PE/EA=100/1 to 1/1) to give 6_X01′Y01a (9.4 g) as a white solid.

LCMS: m/z=377.3 [M+1]⁺

### Example 1-8

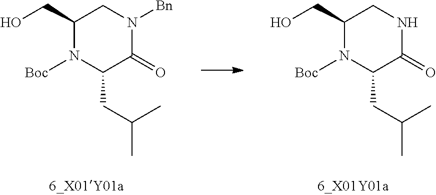 [SMILES: CC(C)C[C@H]1C(=O)N(Cc2ccccc2)C[C@H](CO)N1C(=O)OC(C)(C)C ; CC(C)C[C@H]1C(=O)NC[C@H](CO)N1C(=O)OC(C)(C)C]

NH₃ (gas) was added to a flask at −60° C. to give a solution of NH₃ (50 mL). Then Li (1.3 g) was added to the solution at −78° C. in portions and then stirred at −78° C. for 10 min. Then a solution of 6_X01′Y01a (4.0 g) in THE (32 mL) was added to the mixture at −78° C. and stirred at −78° C. for 2 hr. The mixture was poured into sat·NH₄Claq. (0.50 L) and then extracted with EtOAc (0.10 L×3). The combined organic layers were washed with brine (0.10 L×2), dried over Na₂SO₄, filtered, and concentrated under reduced pressure to give a residue. The residue was purified by column chromatography (SiO₂, PE/EA=10/1 to 1/1) to give 6_X01Y01a (2.1 g) as a white solid.

LCMS: m/z=309.1 [M+Na+]+

### Example 1-9

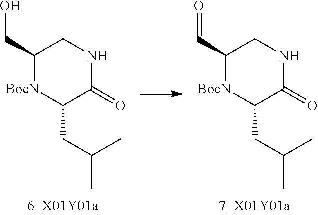 [SMILES: CC(C)C[C@H]1C(=O)NC[C@H](CO)N1C(=O)OC(C)(C)C ; CC(C)C[C@H]1C(=O)NC[C@H](C=O)N1C(=O)OC(C)(C)C]

To a solution of DMSO (2.1 g) in DCM (30 mL) was added (COCl)₂ (2.0 g) at −70° C., the mixture was stirred at −70° C. for 1 hr. Then a solution of 6_X01Y01a (3.0 g) in DCM (30 mL) was added dropwise at −70° C. After addition, DIPEA (6.8 g) was added dropwise at −60° C. The mixture was allowed to warm to 20° C. and stirred for 1 hr. The reaction mixture was washed with 1 N HCl (40 mL×2), and then washed with sat·NaHCO₃aq. (40 mL×2) and brine (40 mL×2). The organic layer was dried over Na₂SO₄, filtered, and concentrated under reduced pressure to give a residue. The residue was purified by column chromatography (SiO₂, PE/EA=20/1 to 0/1) to give 7_X01Y01a (1.8 g) as yellow oil.

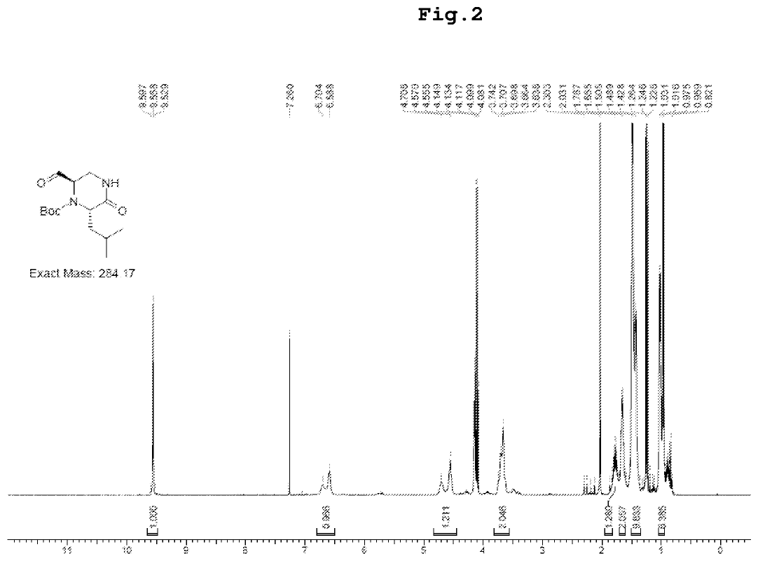

¹H NMR (400 MHZ, CDCl₃) data of 7_X01Y01a is shown in FIG. 2.

### Example 1-10

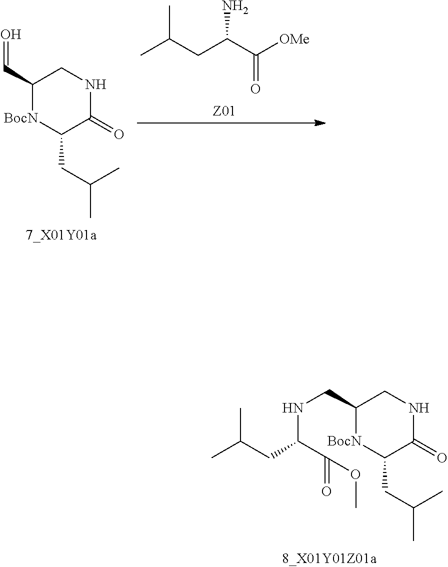

To a solution of Z01 (1.4 g, HCl salt) in MeOH (20 mL) was added NaOAc (1.0 g) and the mixture was stirred at 20° C. for 0.5 hr. Then 7_X01Y01a (1.8 g) was added and the mixture was stirred at 20° C. for 0.5 hr. To the mixture was added NaBH₃CN (0.80 g) and the mixture was stirred at 20° C. for 15 hr. The mixture was concentrated under reduced pressure to give a residue. The residue was diluted with EtOAc (50 mL) and washed with sat·NaHCO₃aq. (30 mL×2), brine (20 mL×2), dried over Na₂SO₄, filtered, and concentrated under reduced pressure to give a residue. The residue was purified by prep-HPLC to give 8_X01Y01Z01a (0.7 g, TFA salt) as a white solid.

LCMS: m/z=414.3 [M+1]+

### Example 1-11

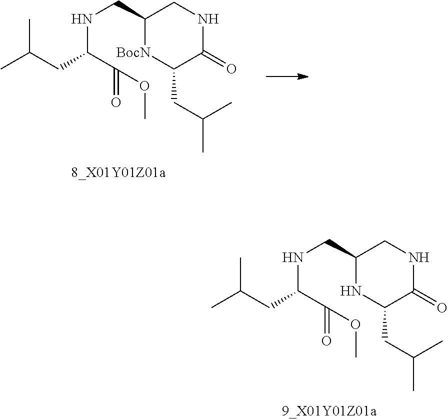 [SMILES: COC(=O)[C@H](CC(C)C)NC[C@H]1CNC(=O)[C@H](CC(C)C)N1C(=O)OC(C)(C)C ; COC(=O)[C@H](CC(C)C)NC[C@H]1CNC(=O)[C@H](CC(C)C)N1]

To a solution of 8_X01Y01Z01a (0.40 g) in DCM (3.0 mL) was added TFA (3.0 mL). The mixture was stirred at 20° C. for 2 hr. The residue was concentrated below 30° C. to give a residue. The reaction mixture was diluted with 20% K₂CO₂aq. (20 mL) and extracted with CHCl₃ (20 mL×2). The organic layers were dried over Na₂SO₄, filtered, and concentrated under reduced pressure to give 9_X01Y01Z01a (0.25 g) as a white solid.

LCMS: m/z=314.2 [M+1]+

### Example 1-12

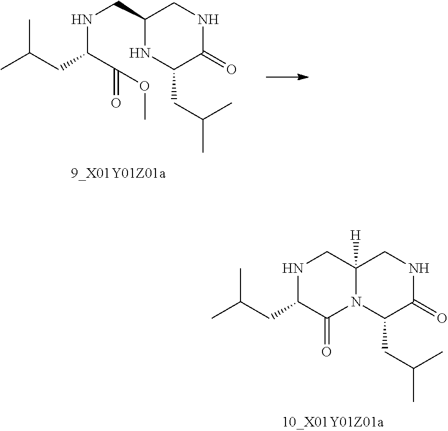 [SMILES: COC(=O)[C@H](CC(C)C)NC[C@H]1CNC(=O)[C@H](CC(C)C)N1 ; CC(C)C[C@@H]1NC[C@H]2CNC(=O)[C@H](CC(C)C)N2C1=O]

To a solution of 9_X01Y01Z01a (0.25 g) in MeCN (2.0 mL) was added AcOH (2.0 mL) and the mixture was stirred at 50° C. for 2 hr. The reaction mixture was concentrated under reduced pressure to give a residue. The residue was purified by prep-HPLC to give 10_X01Y01Z01a (0.20 g) as a white solid.

LCMS: m/z=282.2 [M+1]+

### Example 1-13

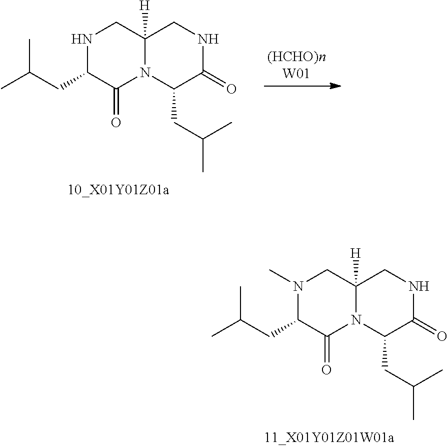 [SMILES: CC(C)C[C@@H]1NC[C@H]2CNC(=O)[C@H](CC(C)C)N2C1=O ; CC(C)C[C@H]1C(=O)N2[C@H](CNC(=O)[C@@H]2CC(C)C)CN1C]

To a solution of 10_X01Y01Z01a (60 mg) in MeOH (2.0 mL) was added NaOAc (25 mg) and the mixture was stirred at 20° C. for 0.5 hr. Then W01 (8.4 μL) was added and the mixture was stirred at 20° C. for 0.5 hr. To the mixture was added NaBH₃CN (29 mg) and the mixture was stirred at 20° C. for 15 hr. The mixture was quenched by addition of water (0.10 mL) and then filtered. The filtrate was purified by prep-HPLC to give 11_X01Y01Z01W01a (29 mg) as an off-white solid.

[Example 2] Synthesis of (3S, 6S, 9aR)-2-acetyl-3,6-diisobutylhexahydro-4H-pyrazino[1,2-a]pyrazine-4,7(6H)-dione (11_X01Y01Z01W02a)

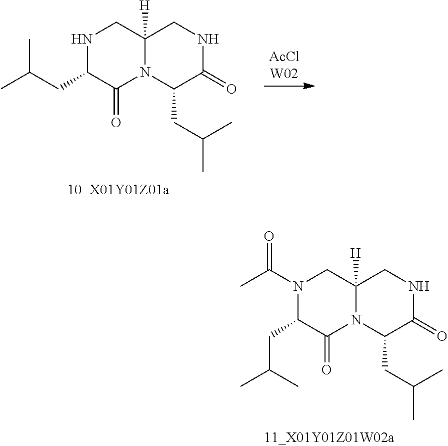 [SMILES: CC(C)C[C@@H]1NC[C@H]2CNC(=O)[C@H](CC(C)C)N2C1=O ; CC(=O)N1C[C@H]2CNC(=O)[C@H](CC(C)C)N2C(=O)[C@@H]1CC(C)C]

To a solution of 10_X01Y01Z01a (60 mg) in DCM (1.0 mL) was added TEA (46 mg) and W02 (13 mg) at 0° C. The mixture was stirred at 20° C. for 16 hr. The reaction mixture was concentrated under reduced pressure to give a residue. The residue was purified by prep-HPLC to give 11_X01Y01Z01W02a (31 mg) as a white solid.

[Example 3] Synthesis of (3S,6S,9aR)-2-(3-(4-hydroxyphenyl)propanoyl)-3,6-diisobutylhexahydro-4H-pyrazino[1,2-a]pyrazine-4,7(6H)-dione (11_X01Y01Z01W08a)

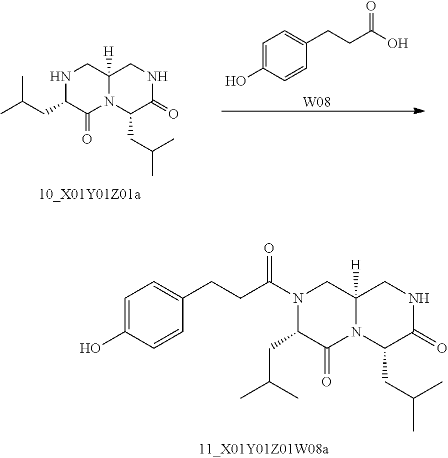

To a solution of 10_X01Y01Z01a (44 mg) and W08 (31 mg) in DMF (2.0 mL) was added DIPEA (82 μL) and HATU (72 mg) at 0° C. The mixture was stirred at 20° C. for 16 hr. The reaction mixture was filtered. The filtrate was purified by prep-HPLC to give 11_X01Y01Z01W08a (34 mg) as a white solid.

[Example 4] Synthesis of 3-((3S,6S,9aR)-6-isobutyl-2-(4-methylpentyl)-4,7-dioxooctahydro-2H-pyrazino[1,2-a]pyrazin-3-yl)propenamide (11_X01Y01Z06W03a)

### Example 4-1

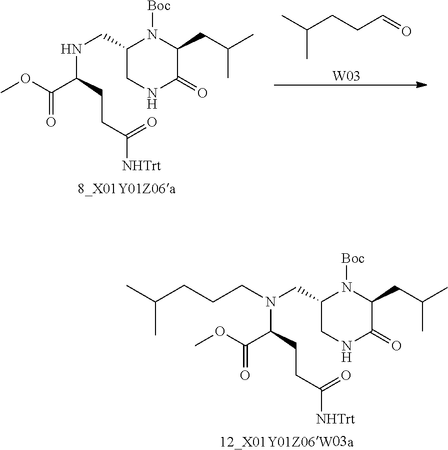

A mixture of W03 (90 mg) and 8_X01Y01Z06′a (0.12 g) in MeOH (2.0 mL) was stirred at 20° C. for 0.5 hr. Then to the reaction mixture was added NaBH₃CN (34 mg). The mixture was stirred at 40° C. for 16 hr. The reaction mixture was concentrated under reduced pressure to give a residue. The residue was purified by column chromatography (SiO₂, PE/EA=50/1 to 1/1) to give 12_X01Y01Z06′W03a (0.15 g) as yellow oil.

LCMS: m/z=756.4 [M+1]+

### Example 4-2

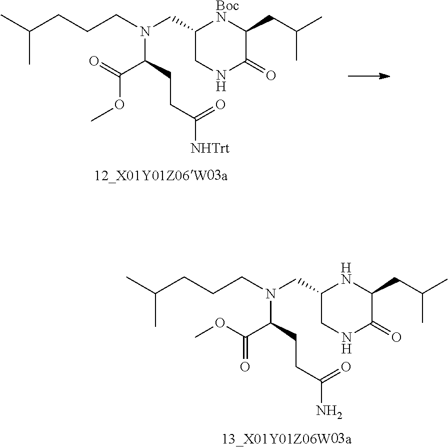

To a solution of 12_X01Y01Z06′W03a (0.15 g) in DCM (2.0 mL) was added TFA (2.0 mL). The mixture was stirred at 20° C. for 2 hr. The reaction mixture was concentrated under reduced pressure to give 13_X01Y01Z06W03a (80 mg) as yellow oil.

LCMS: m/z=413.4 [M+1]+

### Example 4-3

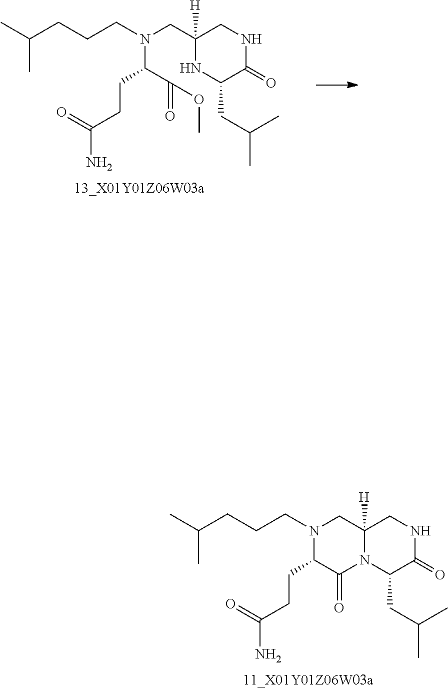

To a solution of 13_X01Y01Z06W03a (80 mg) in MeCN (2.0 mL) was added AcOH (2.0 mL). The mixture was stirred at 60° C. for 2 hr. The reaction mixture was concentrated under reduced pressure to give a residue. The residue was purified by prep-HPLC to give 11_X01Y01Z06W03a (30 mg) as a white solid.

[Example 39] Synthesis of (6S,10aR)-6-benzyl-8-isopentyl-2-(3-phenylpropanoyl)hexahydropyrazino[1,2-d][1,4]diazepine-4,7(1H,6H)-dione (11_X02Y02Z07W07b)

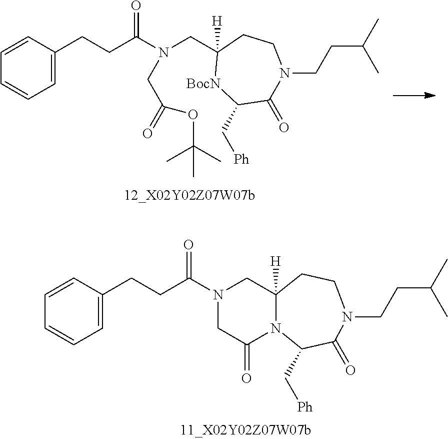 [SMILES: CC(C)CCN1CC[C@H](CN(CC(=O)OC(C)(C)C)C(=O)CCc2ccccc2)N(C(=O)OC(C)(C)C)[C@@H](Cc2ccccc2)C1=O ; CC(C)CCN1CC[C@@H]2CN(C(=O)CCc3ccccc3)CC(=O)N2[C@@H](Cc2ccccc2)C1=O]

To a solution of 12_X02Y02Z07W07b (5.8 mg) in DCM (0.2 mL) was added TFA (0.1 mL) at rt. After being stirred at same temperature for 3 hr, the reaction mixture was concentrated in vacuo. The residue was dissolved in dichloromethane (0.2 mL), and was added DIPEA (3.9 μL) and HATU (4.1 mg) at rt. After being stirred at same temperature for 5 hr, the reaction was quenched with 1 M HClaq. and the aqueous layer was extracted with two portions of EtOAc. The combined organic layers were washed with sat·NaHCO₃aq. and brine, dried over magnesium sulfate, filtered, and concentrated in vacuo. The residue was purified by preparative layer plates (60% EtOAc in hexane) to give 11_X02Y02Z07W07b (3.9 mg) as colorless oil. .

[Reference Example 1] (Example of alternative methods for the reactions in Example 1-3)

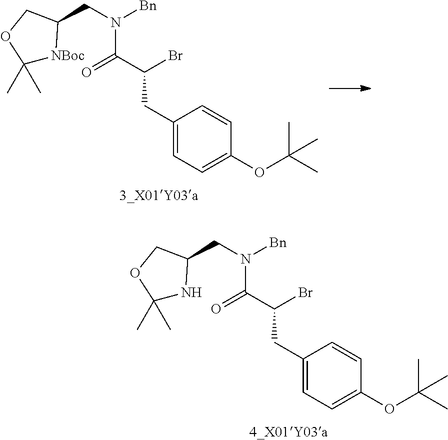 [SMILES: CC(C)(C)OC(=O)N1[C@H](CN(Cc2ccccc2)C(=O)[C@H](Br)Cc2ccc(OC(C)(C)C)cc2)COC1(C)C ; CC(C)(C)Oc1ccc(C[C@@H](Br)C(=O)N(Cc2ccccc2)C[C@@H]2COC(C)(C)N2)cc1]

To a solution of 3_X01′Y03′a (80 g) and TEA (0.28 L) in DCM (1.2 L) was added TMSOTf (0.24 L) dropwise at 0-10° C. The mixture was stirred at 20° C. for 1 hr. The mixture was washed with water (1.5 L×2), dried over Na₂SO₄, filtered, and concentrated to give 4_X01′Y03′a (80 g) as yellow oil.

LCMS: m/z=505.2 [M+2]+, 503.2[M]+

### [Reference Example 2] Synthesis of Intermediate 6_X01″Y04′a

### Reference Example 2-1

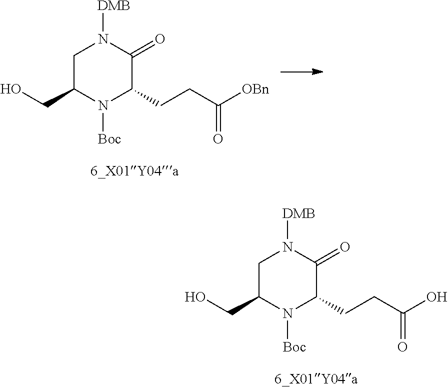

To a solution of 6_X01″Y04″′a (4.0 g) in MeOH (40 mL) was added Pd/C (0.40 g, 10% purity) under N₂ atmosphere. The suspension was degassed and purged with H₂ for 3 times. The mixture was stirred under H₂ (15 Psi) at 23° C. for 2 hr. The reaction mixture was filtered and concentrated under reduced pressure to give a residue. This reaction was performed two batches to give 6_X01″Y04″a (5.5 g) as colorless oil.

LCMS: m/z=453.0 [M+1]+

### Reference Example 2-2

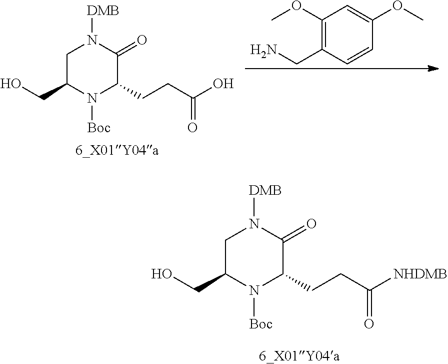

To a solution of 6_X01″Y04″a (5.0 g) and (2,4-dimethoxyphenyl)methanamine (3.3 g) in DCM (50 mL) was added HOBt (2.7 g), then 2,4,6-trimethy pyridine (2.4 g) at 0 to 10° C. The mixture was stirred at 0° C. for 0.5 hr, and then EDCI (3.8 g) was added to the mixture at 0° C. The mixture was stirred at 20° C. for 15 hr. The reaction mixture was diluted with water (0.10 L). The organic layer was washed with 1 N HCl (0.10 L×2) , then washed with sat·Na₂CO₃aq. (0.10 L×2), dried over Na₂SO₄, filtered, and concentrated under reduced pressure to give a residue. The residue was purified by column chromatography (SiO₂, PE/EA=5/1 to 0/1) to give 6_X01″Y04′a (5.0 g) as colorless oil.

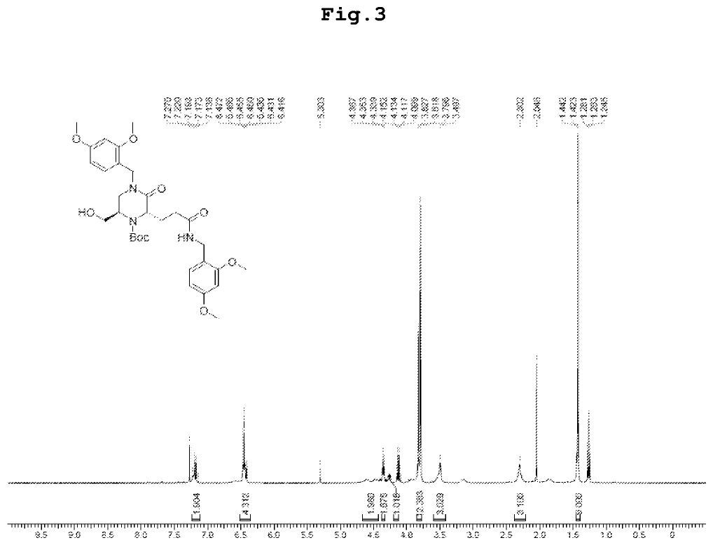

¹H NMR (400 MHZ, CDCl₃) data of 6_X01″Y04′a is shown in FIG. 3.

[Reference Example 3] (Example of alternative methods for the reactions in Example 1-9)

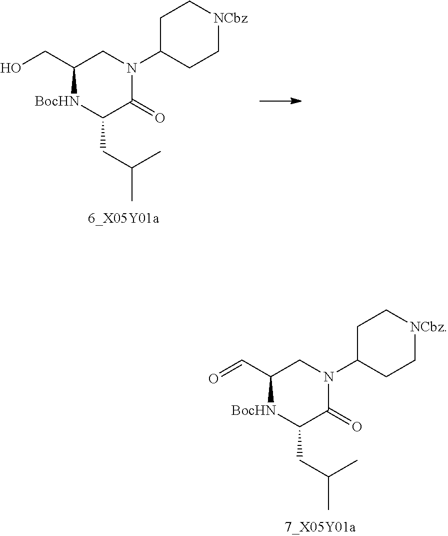

6_X05Y01a (0.25 g), AZADOL (0.80 mg), and KBr (5.9 mg) were dissolved in DCM (3.0 mL) and sat·NaHCO₃aq. (1.0 mL) mixed solvent. The mixture was stirred and cooled with an ice bath, and then 5% NaClOaq. (0.62 mL) in sat·NaHCOsaq. (1.0 mL) was added dropwise. After stirring for 0.5 hr, Na₂SO₃ (0.13 g) in water (1.0 mL) was added. The mixture was transferred to a separating funnel with DCM (10 mL) and water (5 mL). The mixture was partitioned, and the water phase was extracted with DCM (10 mL×2). The combined organic layer was dried over Na₂SO₄ and concentrated under reduced pressure. The residue was purified by column chromatography (SiO₂, Hexane/EtOAc=95/5 to 40/60) to give 7_X05Y01a (0.22 g) as light-yellow oil.

LCMS: m/z=524.3 [M+Na+]+

### Examples 5-38, 40-43

Example compounds 5-38, 40-43 shown in Table 6 were synthesized via various intermediates shown in Tables 4 and 5 and by appropriately selecting the reagents shown in Table 3 in each step of the above-mentioned Examples 1-4, 39 and Reference Examples 1-3.

The structures, names, and the MS data of the Example compounds are shown in Table 7.

TABLE 3
Reagents 1, Xi, Yj, Zm, Wn

| Structure | 1 |
|---|---|
| 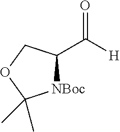 | 1a |
| 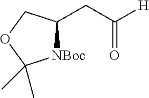 | 1b |
| Structure | Xi |
| 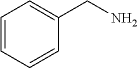 | X01′ |
| 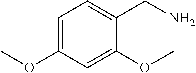 | X01″ |
| 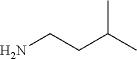 | X02 |
| 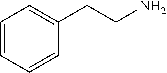 | X03 |
| 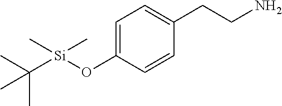 [SMILES: CC(C)(C)[Si](C)(C)Oc1ccc(CCN)cc1] | X04′ |
| 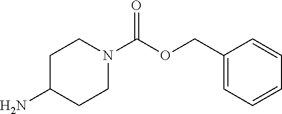 [SMILES: NC1CCN(C(=O)OCc2ccccc2)CC1] | X06 |
| Structure | Yj |
| 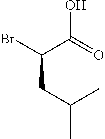 | Y01 |
| 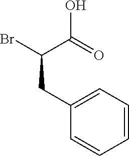 [SMILES: O=C(O)[C@H](Br)Cc1ccccc1] | Y02 |
| 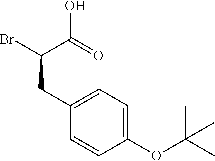 [SMILES: CC(C)(C)Oc1ccc(C[C@@H](Br)C(=O)O)cc1] | Y03′ |
| 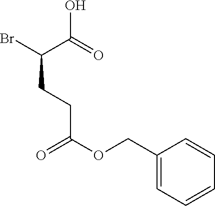 [SMILES: O=C(CC[C@@H](Br)C(=O)O)OCc1ccccc1] | Y04′′′ |
| Structure | Zm |
| 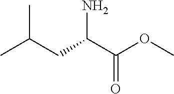 | Z01 |
| 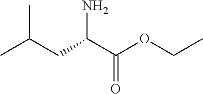 | Z01 (Et) |
| 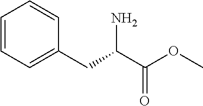 | Z02 |
| 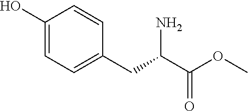 | Z03 |
| 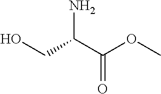 | Z04 |
| 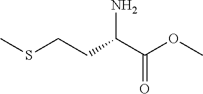 | Z05 |
| 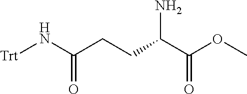 | Z06′ |
| 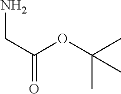 | Z07 |
| Structure | Wn |
| (HCHO)ₙ | W01 |
| 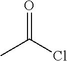 | W02 |
| 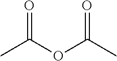 | W02 (Ac₂O) |
| 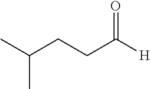 | W03 |
| 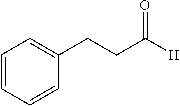 | W04 |
| 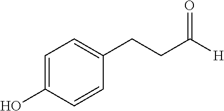 [SMILES: O=CCCc1ccc(O)cc1] | W05 |
| 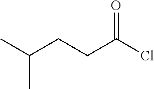 | W06 |
| 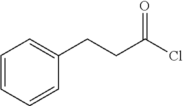 [SMILES: O=C(Cl)CCc1ccccc1] | W07 |
| 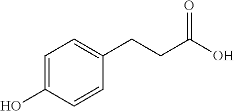 [SMILES: O=C(O)CCc1ccc(O)cc1] | W08 |
| none (W09: R4 = H, V = bond) | W09 |
| 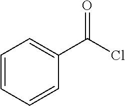 | W10 |

TABLE 4
Compounds 2_Xi

| | Compound | Starting material | |
|---|---|---|---|
| Structure | 2_Xi | 1 | Xi |
| 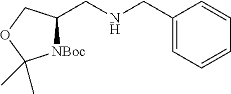 | 2_X01′a | 1a | X01′ |
| 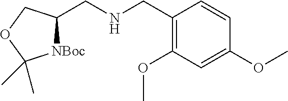 | 2_X01″a | 1a | X01″ |
| 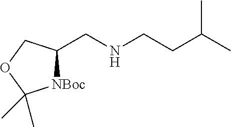 | 2_X02a | 1a | X02 |
| 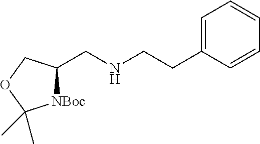 | 2_X03a | 1a | X03 |
| 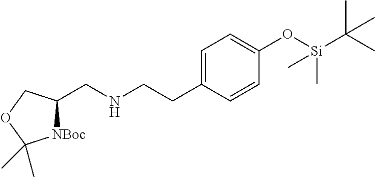 | 2_X04′a | 1a | X04′ |
| 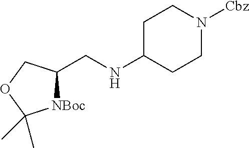 | 2_X05a | 1a | X05 |
| 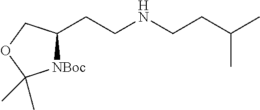 | 2_X02b | 1b | X02 |

TABLE 5
Intermediates 7_XiYj

| | Compound | Starting material | |
|---|---|---|---|
| Structure | 7_XiYj | 2_Xi | Yj |
| 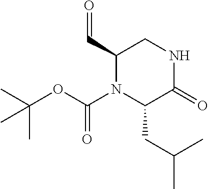 [SMILES: CC(C)C[C@H]1C(=O)NC[C@H](C=O)N1C(=O)OC(C)(C)C] | 7_X01Y01a | 2_X01′a | Y01 |
| 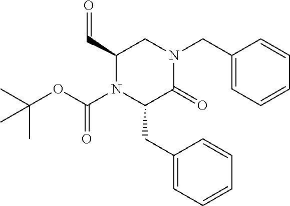 [SMILES: CC(C)(C)OC(=O)N1[C@@H](C=O)CN(Cc2ccccc2)C(=O)[C@@H]1Cc1ccccc1] | 7_X01′Y02a | 2_X01′a | Y02 |
| 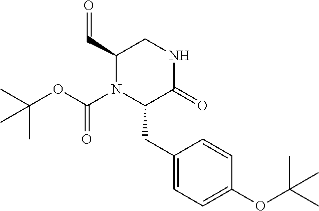 [SMILES: CC(C)(C)OC(=O)N1[C@@H](C=O)CNC(=O)[C@@H]1Cc1ccc(OC(C)(C)C)cc1] | 7_X01Y03′a | 2_X01′a | Y03′ |
| 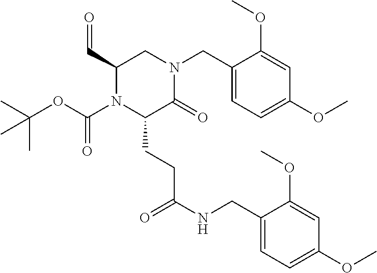 [SMILES: COc1ccc(CNC(=O)CC[C@H]2C(=O)N(Cc3ccc(OC)cc3OC)C[C@H](C=O)N2C(=O)OC(C)(C)C)c(OC)c1] | 7y_X01″Y04′a | 2_X01″a | Y04′′′ |
| 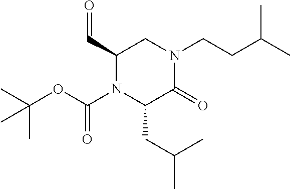 [SMILES: CC(C)CCN1C[C@H](C=O)N(C(=O)OC(C)(C)C)[C@@H](CC(C)C)C1=O] | 7_X02Y01a | 2_X02a | Y01 |
| 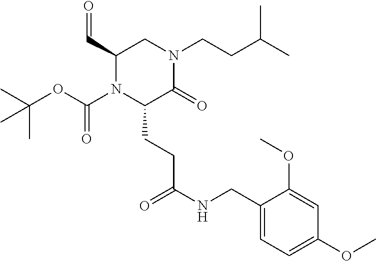 [SMILES: COc1ccc(CNC(=O)CC[C@H]2C(=O)N(CCC(C)C)C[C@H](C=O)N2C(=O)OC(C)(C)C)c(OC)c1] | 7_X02Y04′a | 2_X02a | Y04′′′ |
| 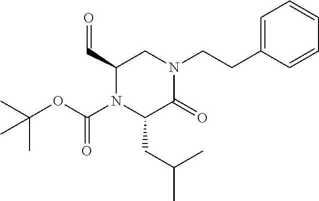 [SMILES: CC(C)C[C@H]1C(=O)N(CCc2ccccc2)C[C@H](C=O)N1C(=O)OC(C)(C)C] | 7_X03Y01a | 2_X03a | Y01 |
| 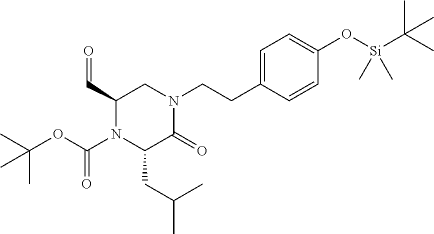 [SMILES: CC(C)C[C@H]1C(=O)N(CCc2ccc(O[Si](C)(C)C(C)(C)C)cc2)C[C@H](C=O)N1C(=O)OC(C)(C)C] | 7_X04Y01a | 2_X04′a | Y01 |
| 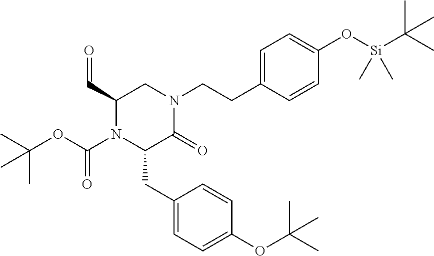 [SMILES: CC(C)(C)OC(=O)N1[C@@H](C=O)CN(CCc2ccc(O[Si](C)(C)C(C)(C)C)cc2)C(=O)[C@@H]1Cc1ccc(OC(C)(C)C)cc1] | 7_X04′Y03′a | 2_X04′a | Y03′ |
| 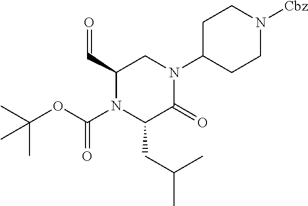 [SMILES: CC(C)C[C@H]1C(=O)N(C2CCN(C(=O)OCc3ccccc3)CC2)C[C@H](C=O)N1C(=O)OC(C)(C)C] | 7_X05Y01a | 2_X05a | Y01 |
| 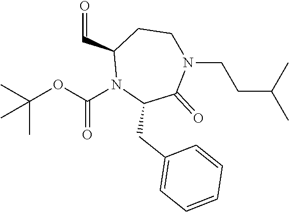 [SMILES: CC(C)CCN1CC[C@H](C=O)N(C(=O)OC(C)(C)C)[C@@H](Cc2ccccc2)C1=O] | 7_X02Y02b | 2_X02b | Y02 |

TABLE 6

| Example | Compound | Starting material | | |
|---|---|---|---|---|
| Nos . | 11_XiYjZmWn | 7_XiYj | Zm | Wn |
| 1 | 11_X01Y01Z01W01a | 7_X01Y01a | Z01 | W01 |
| 2 | 11_X01Y01Z01W02a | 7_X01Y01a | Z01 | W02 |
| 3 | 11_X01Y01Z01W08a | 7_X01Y01a | Z01 | W08 |
| 4 | 11_X01Y01Z06W03a | 7_X01Y01a | Z06′ | W03 |
| 5 | 11_X01Y01Z01W03a | 7_X01Y01a | Z01 | W03 |
| 6 | 11_X01Y01Z01W04a | 7_X01Y01a | Z01 | W04 |
| 7 | 11_X01Y01Z01W05a | 7_X01Y01a | Z01 | W05 |
| 8 | 11_X01Y01Z02W03a | 7_X01Y01a | Z02 | W03 |
| 9 | 11_X01Y01Z03W03a | 7_X01Y01a | Z03 | W03 |
| 10 | 11_X01Y01Z04W03a | 7_X01Y01a | Z04 | W03 |
| 11 | 11_X01Y01Z05W03a | 7_X01Y01a | Z05 | W03 |
| 12 | 11_X01Y01Z06W06a | 7_X01Y01a | Z06′ | W06 |
| 13 | 11_X01Y02Z01W03a | 7_X01′Y02a | Z01 | W03 |
| 14 | 11_X01Y02Z01W05a | 7_X01′Y02a | Z01 | W05 |
| 15 | 11_X01Y02Z01W07a | 7_X01′Y02a | Z01 | W07 |
| 16 | 11_X01Y03Z01W04a | 7_X01Y03′a | Z01 | W04 |
| 17 | 11_X01Y03Z01W05a | 7_X01Y03′a | Z01 | W05 |
| 18 | 11_X01Y04Z01W06a | 7_X01′′Y04′a | Z01 | W06 |
| 19 | 11_X02Y01Z01W03a | 7_X02Y01a | Z01 | W03 |
| 20 | 11_X02Y01Z01W06a | 7_X02Y01a | Z01 | W06 |
| 21 | 11_X02Y01Z02W03a | 7_X02Y01a | Z02 | W03 |
| 22 | 11_X02Y01Z02W04a | 7_X02Y01a | Z02 | W04 |
| 23 | 11_X02Y01Z02W05a | 7_X02Y01a | Z02 | W05 |
| 24 | 11_X02Y01Z02W06a | 7_X02Y01a | Z02 | W06 |
| 25 | 11_X02Y01Z02W08a | 7_X02Y01a | Z02 | W08 |
| 26 | 11_X02Y01Z03W01a | 7_X02Y01a | Z03 | W01 |
| 27 | 11_X02Y01Z03W02a | 7_X02Y01a | Z03 | W02 |
| 28 | 11_X02Y01Z04W01a | 7_X02Y01a | Z04 | W01 |
| 29 | 11_X02Y01Z05W01a | 7_X02Y01a | Z05 | W01 |
| 30 | 11_X02Y01Z06W02a | 7_X02Y01a | Z06′ | W02 |
| 31 | 11_X02Y04Z01W01a | 7_X02Y04′a | Z01 | W01 |
| 32 | 11_X02Y04Z01W02a | 7_X02Y04′a | Z01 | W02 |
| 33 | 11_X03Y01Z01W03a | 7_X03Y01a | Z01 | W03 |
| 34 | 11_X03Y01Z01W06a | 7_X03Y01a | Z01 | W06 |
| 35 | 11_X04Y01Z01W03a | 7_X04′Y01a | Z01 | W03 |
| 36 | 11_X04Y01Z01W06a | 7_X04′Y01a | Z01 | W06 |
| 37 | 11_X04Y03Z01W01a | 7_X04′Y03′a | Z01 | W01 |
| 38 | 11_X05Y01Z01W09a | 7_X05Y01a | Z01 (Et) | W09 |
| 39 | 11_X02Y02Z07W07b | 7_X02Y02b | Z07 | W07 |
| 40 | 11_X02Y02Z02W09b | 7_X02Y02b | Z02 | W09 |
| 41 | 11_X02Y02Z07W06b | 7_X02Y02b | Z07 | W06 |
| 42 | 11_X02Y02Z02W10b | 7_X02Y02b | Z02 | W10 |
| 43 | 11_X02Y02Z02W02b | 7_X02Y02b | Z02 | W02 (Ac₂O) |

TABLE 7-1

| Example | | | | Observed |
|---|---|---|---|---|
| Nos. | Structure | name | MW | Mass |
| 1 | 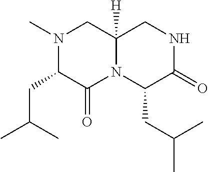 [SMILES: CC(C)C[C@H]1C(=O)N2[C@H](CNC(=O)[C@@H]2CC(C)C)CN1C] | (3S,6S,9aR)-3,6-diisobutyl)-2- methylhexahydro-4H-pyrazino[1,2- a]pyrazine-4,7(6H)-dione | 295.4 | 296.2 |
| 2 | 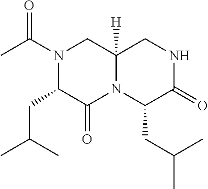 [SMILES: CC(=O)N1C[C@H]2CNC(=O)[C@H](CC(C)C)N2C(=O)[C@@H]1CC(C)C] | (3S,6S,9aR)-2-acetyl-3,6- diisobutylhexahydro-4H-pyrazino[1,2- a]pyrazine-4,7(6H)-dione | 323.4 | 324.2 |
| 3 | 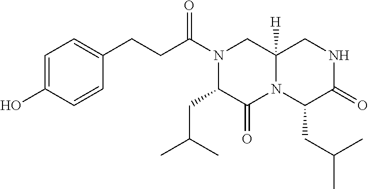 [SMILES: CC(C)C[C@H]1C(=O)N2[C@H](CNC(=O)[C@@H]2CC(C)C)CN1C(=O)CCc1ccc(O)cc1] | (3S,6S,9aR)-2-(3-(4- hydroxyphenyl)propanoyl)-3,6- diisobutylhexahydro-4H-pyrazino[1,2- a]pyrazine-4,7(6H)-dione | 429.6 | 430.2 |
| 4 | 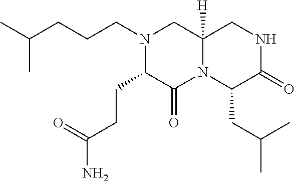 [SMILES: CC(C)CCCN1C[C@H]2CNC(=O)[C@H](CC(C)C)N2C(=O)[C@@H]1CCC(N)=O] | 3-((3S,6S,9aR)-6-isobutyl-2-(4- methylpentyl)-4,7-dioxooctahydro-2H- pyrazino[1,2-a]pyrazin-3-yl)propanamide | 380.5 | 381.2 |
| 5 | 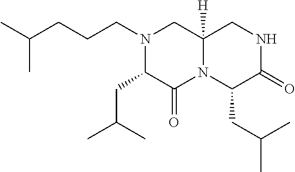 [SMILES: CC(C)CCCN1C[C@H]2CNC(=O)[C@H](CC(C)C)N2C(=O)[C@@H]1CC(C)C] | (3S,6S,9aR)-3,6-diisobutyl-2-(4- methylpentyl)hexahydro-4H-pyrazino[1,2- a]pyrazine-4,7(6H)-dione | 365.6 | 366.3 |
| 6 | 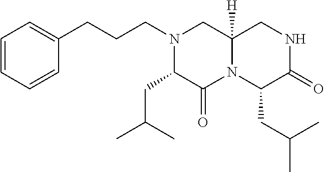 [SMILES: CC(C)C[C@H]1C(=O)N2[C@H](CNC(=O)[C@@H]2CC(C)C)CN1CCCc1ccccc1] | (3S,6S,9aR)-3,6-diisobutyl-2-(3- phenylpropyl)hexahydro-4H-pyrazino[1,2- a]pyrazine-4,7(6H)-dione | 399.6 | 400.3 |
| 7 | 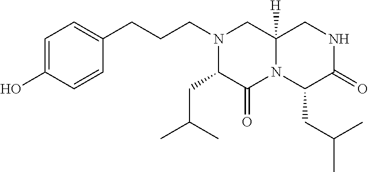 [SMILES: CC(C)C[C@H]1C(=O)N2[C@H](CNC(=O)[C@@H]2CC(C)C)CN1CCCc1ccc(O)cc1] | (3S,6S,9aR)-2-(3-(4- hydroxyphenyl)propyl)-3,6- diisobutylhexahydro-4H-pyrazino[1,2- a]pyrazine-4,7(6H)-dione | 415.6 | 416.3 |
| 8 | 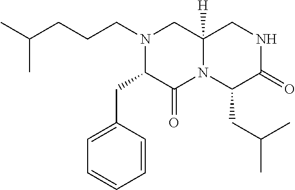 [SMILES: CC(C)CCCN1C[C@H]2CNC(=O)[C@H](CC(C)C)N2C(=O)[C@@H]1Cc1ccccc1] | (3S,6S,9aR)-3-benzyl-6-isobutyl-2-(4- methylpenyl)hexahydro-4H-pyrazino[1,2- a]pyrazine-4,7(6H)-dione | 399.6 | 400.3 |
| 9 | 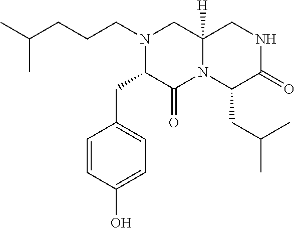 [SMILES: CC(C)CCCN1C[C@H]2CNC(=O)[C@H](CC(C)C)N2C(=O)[C@@H]1Cc1ccc(O)cc1] | (3S,6S,9aR)-3-(4-hydroxybenzyl)-6- isobutyl-2-(4-methylpentyl)hexahydro- 4H-pyrazino[1,2-a]pyrazine-4,7(6H)-dione | 415.6 | 416.3 |
| 10 | 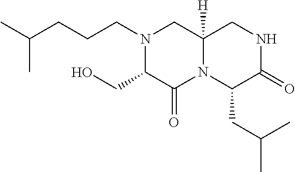 [SMILES: CC(C)CCCN1C[C@H]2CNC(=O)[C@H](CC(C)C)N2C(=O)[C@@H]1CO] | (3S,6S,9aR)-3-(hydroxymethyl)-6- isobutyl-2-(4-methylpentyl)hexahydro- 4H-pyrazino[1,2-a]pyrazine-4,7(6H)-dione | 339.5 | 340.3 |
| 11 | 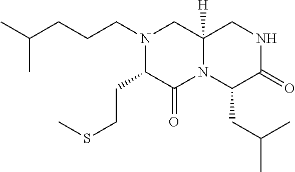 [SMILES: CSCC[C@H]1C(=O)N2[C@H](CNC(=O)[C@@H]2CC(C)C)CN1CCCC(C)C] | (3S,6S,9aR)-6-isobutyl-2-(4- methylpentyl)-3-(2- (methylthio)ethyl)hexahydro-4H- pyrazino[1,2-a]pyrazine-4,7(6H)-dione | 383.6 | 384.2 |
| 12 | 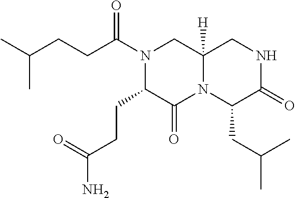 [SMILES: CC(C)CCC(=O)N1C[C@H]2CNC(=O)[C@H](CC(C)C)N2C(=O)[C@@H]1CCC(N)=O] | 3-((3S,6S,9aR)-6-isobutyl-2-(4- methylpentanoyl)-4,7-dioxooctahydro-2H- pyrazino[1,2-a]pyrazin-3-yl)propanamide | 394.5 | 395.2 |
| 13 | 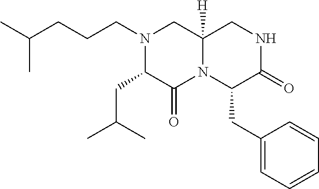 [SMILES: CC(C)CCCN1C[C@H]2CNC(=O)[C@H](Cc3ccccc3)N2C(=O)[C@@H]1CC(C)C] | (3S,6S,9aR)-6-benzyl-3-isobutyl-2-(4- methylpentyl)hexahydro-4H-pyrazino[1,2- a]pyrazine-4,7(6H)-dione | 399.6 | 400.3 |
| 14 | 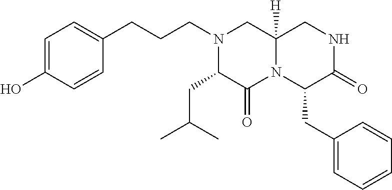 [SMILES: CC(C)C[C@H]1C(=O)N2[C@H](CNC(=O)[C@@H]2Cc2ccccc2)CN1CCCc1ccc(O)cc1] | (3S,6S,9aR)-6-benzyl-2-(3-(4- hydroxyphenyl)propyl)-3- isobutylhexahydro-4H-pyrazino[1,2- a]pyrazine-4,7(6H)-dione | 449.6 | 450.2 |
| 15 | 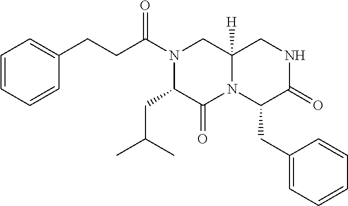 [SMILES: CC(C)C[C@H]1C(=O)N2[C@H](CNC(=O)[C@@H]2Cc2ccccc2)CN1C(=O)CCc1ccccc1] | (3S,6S,9aR)-6-benzyl-3-isobutyl-2-(3- phenylpropanyl)hexahydro-4H- pyrazino[1,2-a]pyrazine-4,7(6H)-dione | 447.6 | 448.2 |
| 16 | 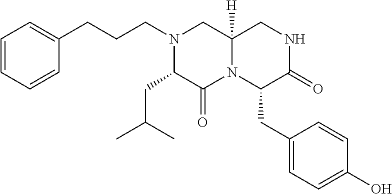 [SMILES: CC(C)C[C@H]1C(=O)N2[C@H](CNC(=O)[C@@H]2Cc2ccc(O)cc2)CN1CCCc1ccccc1] | (3S,6S,9aR)-6-(4-hydroxybenzyl)-3- isobutyl-2-(3-phenylpropyl)hexahydro- 4H-pyrazino[1,2-a]pyrazine-4,7(6H)-dione | 449.6 | 450.3 |
| 17 | 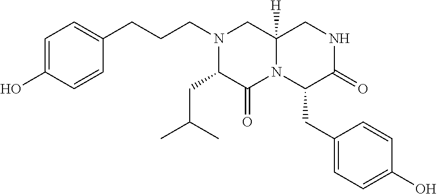 [SMILES: CC(C)C[C@H]1C(=O)N2[C@H](CNC(=O)[C@@H]2Cc2ccc(O)cc2)CN1CCCc1ccc(O)cc1] | (3S,6S,9aR)-6-(4-hydroxybenzyl)-2-(3-(4- hydroxyphenyl)propyl)-3- isobutylhexahydro-4H-pyrazino[1,2- a]pyrazine-4,7(6H)-dione | 465.6 | 466.2 |
| 18 | 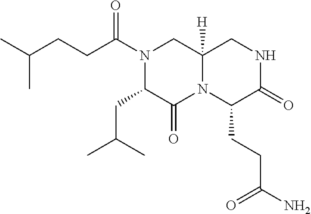 [SMILES: CC(C)CCC(=O)N1C[C@H]2CNC(=O)[C@H](CCC(N)=O)N2C(=O)[C@@H]1CC(C)C] | 3-((4S,7S,9aR)-7-isobutyl-8-(4- methylbentanoyl)-3,6-dioxooctahydro-2H- pyrazino[1,2-a]pyrazin-4-yl)propanamide | 394.5 | 395.2 |
| 19 | 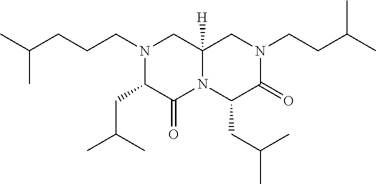 [SMILES: CC(C)CCCN1C[C@H]2CN(CCC(C)C)C(=O)[C@H](CC(C)C)N2C(=O)[C@@H]1CC(C)C] | (3S,6S,9aS)-3,6-diisobutyl-8-isopentyl)-2- (4-methylpentyl)hexahydro-4H- pyrazino[1,2-a]pyrazine-4,7(6H)-dione | 435.7 | 436.4 |
| 20 | 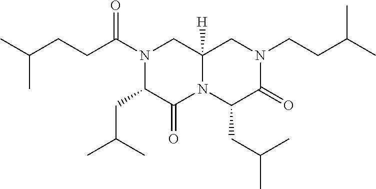 [SMILES: CC(C)CCC(=O)N1C[C@H]2CN(CCC(C)C)C(=O)[C@H](CC(C)C)N2C(=O)[C@@H]1CC(C)C] | (3S,6S,9aR)-3,6-diisobutyl-8-isopentyl-2- (4-methylpentanoyl)hexahydro-4H- pyrazino[1,2-a]pyrazine-4,7(6H)-dione | 449.7 | 450.3 |
| 21 | 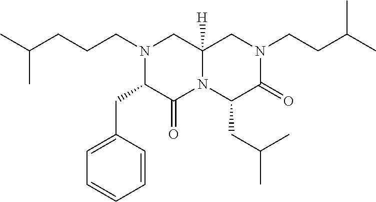 [SMILES: CC(C)CCCN1C[C@H]2CN(CCC(C)C)C(=O)[C@H](CC(C)C)N2C(=O)[C@@H]1Cc1ccccc1] | (3S,6S,9aS)-3-benzyl-6-isobutyl-8- isopentyl-2-(4-methylphenyl)hexahydro- 4H-pyrazino[1,2-a]pyrazine-4,7(6H)-dione | 469.7 | 470.4 |
| 22 | 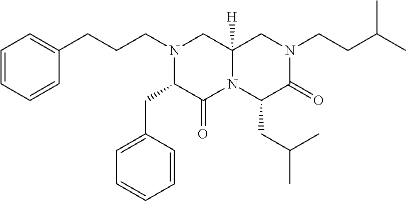 [SMILES: CC(C)CCN1C[C@@H]2CN(CCCc3ccccc3)[C@@H](Cc3ccccc3)C(=O)N2[C@@H](CC(C)C)C1=O] | (3S,6S,9aS)-3-benzyl-6-isobutyl-8- isopentyl-2-(3-phenylpropyl)hexahydro- 4H-pyrazino[1,2-a]pyrazine-4,7(6H)-dione | 503.7 | 504.3 |
| 23 | 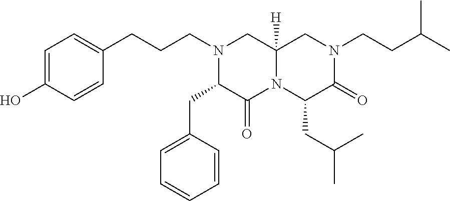 [SMILES: CC(C)CCN1C[C@@H]2CN(CCCc3ccc(O)cc3)[C@@H](Cc3ccccc3)C(=O)N2[C@@H](CC(C)C)C1=O] | (3S,6S,9aS)-3-benzyl-2-(3-(4- hydroxyphenyl)propyl)-6-isobutyl-8- isopentylhexahydro-4H-pyrazino[1,2- a]pyrazine-4,7(6H)-dione | 519.7 | 520.3 |
| 24 | 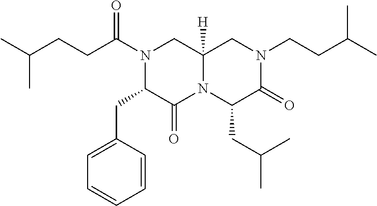 [SMILES: CC(C)CCC(=O)N1C[C@H]2CN(CCC(C)C)C(=O)[C@H](CC(C)C)N2C(=O)[C@@H]1Cc1ccccc1] | (3S,6S,9aR)-3-benzyl-6-isobutyl-8- isopentyl-2-(4- methylpentanoyl)hexahydro-4H- pyrazino[1,2-a]pyrazine-4,7(6H)-dione | 483.7 | 484.3 |
| 25 | 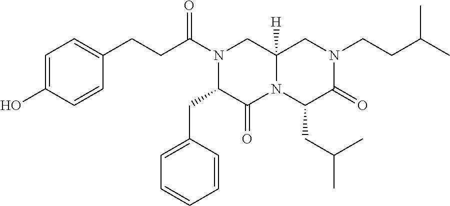 [SMILES: CC(C)CCN1C[C@@H]2CN(C(=O)CCc3ccc(O)cc3)[C@@H](Cc3ccccc3)C(=O)N2[C@@H](CC(C)C)C1=O] | (3S,6S,9aR)-3-benzyl-2-(3-(4- hydroxyphenyl)propanoyl)-6-isobutyl-8- isopentylhexahydro-4H-pyrazino[1,2- a]pyrazine-4,7(6H)-dione | 533.7 | 534.3 |
| 26 | 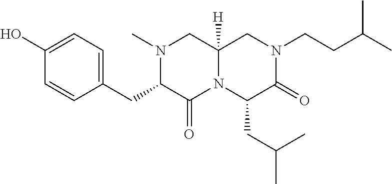 [SMILES: CC(C)CCN1C[C@@H]2CN(C)[C@@H](Cc3ccc(O)cc3)C(=O)N2[C@@H](CC(C)C)C1=O] | (3S,6S,9aS)-3-(4-hydroxybenzyl)-6- isobutyl-8-isopentyl-2-methylhexahydro- 4H-pyrazino[1,2-a]pyrazine-4,7(6H)-dione | 415.6 | 416.3 |
| 27 | 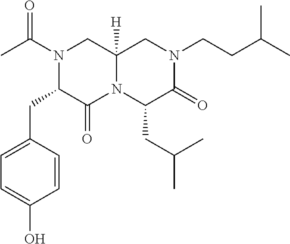 [SMILES: CC(=O)N1C[C@H]2CN(CCC(C)C)C(=O)[C@H](CC(C)C)N2C(=O)[C@@H]1Cc1ccc(O)cc1] | (3S,6S,9aR)-2-acetyl-3-(4- hydroxybenzyl)-6-isobutyl-8- isopentylhexahydro-4H-pyrazino[1,2- a]pyrazine-4,7(6H)-dione | 443.6 | 444.3 |
| 28 | 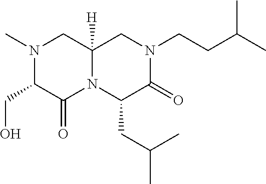 [SMILES: CC(C)CCN1C[C@@H]2CN(C)[C@@H](CO)C(=O)N2[C@@H](CC(C)C)C1=O] | (3S,6S,9aS)-3-(hydroxymethyl)-6- isobutyl-8-isopentyl-2-methylhexahydro- 4H-pyrazino[1,2-a]pyrazine-4,7(6H)-dione | 339.5 | 340.3 |
| 29 | 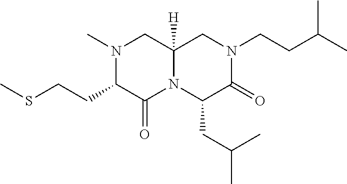 [SMILES: CSCC[C@H]1C(=O)N2[C@H](CN(CCC(C)C)C(=O)[C@@H]2CC(C)C)CN1C] | (3S,6S,9aS)-6-isobutyl-8-isopentyl-2- methyl-3-(2-(methylthio)ethyl)hexahydro- 4H-pyrazino[1,2-a]pyrazine-4,7(6H)-dione | 383.6 | 384.2 |
| 30 | 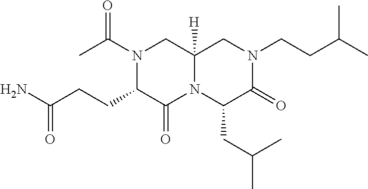 [SMILES: CC(=O)N1C[C@H]2CN(CCC(C)C)C(=O)[C@H](CC(C)C)N2C(=O)[C@@H]1CCC(N)=O] | 3-((3S,6S,9aR)-2-acetyl-6-isobutyl-8- isopentyl-4,7-dioxooctahydro-2H- pyrazino[1,2-a]pyrazin-3-yl)propanamide | 408.5 | 409.3 |
| 31 | 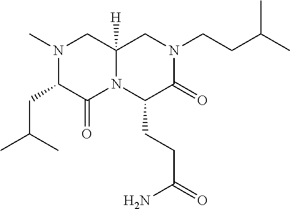 [SMILES: CC(C)CCN1C[C@@H]2CN(C)[C@@H](CC(C)C)C(=O)N2[C@@H](CCC(N)=O)C1=O] | 3-((4S,7S,9aS)-2-7-isobutyl-2-isopentyl-8- methyl-3,6-dioxooctahydro-2H- pyrazino[1,2-a]pyrazin-4-yl)propanamide | 380.5 | 381.3 |
| 32 | 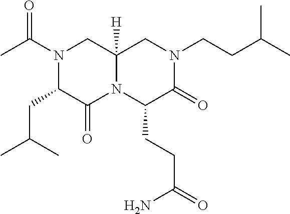 [SMILES: CC(=O)N1C[C@H]2CN(CCC(C)C)C(=O)[C@H](CCC(N)=O)N2C(=O)[C@@H]1CC(C)C] | 3-((4S,7S,9aR)-8-acetyl-7-isobutyl-2- isopentyl-3,6-dioxooctahydro-2H- pyrazino[1,2-a]pyrazin-4-yl)propanamide | 408.5 | 409.3 |
| 33 | 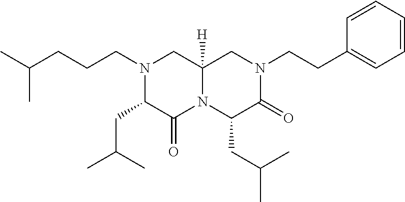 [SMILES: CC(C)CCCN1C[C@H]2CN(CCc3ccccc3)C(=O)[C@H](CC(C)C)N2C(=O)[C@@H]1CC(C)C] | (3S,6S,9aS)-3,6-diisobutyl-2-(4- methylpentyl)-8-phenethylhexahydro-5H- pyrazino[1,2-a]pyrazine-4,7(6H)-dione | 469.7 | 470.4 |
| 34 | 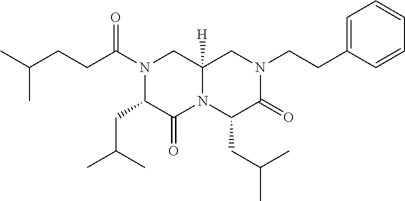 [SMILES: CC(C)CCC(=O)N1C[C@H]2CN(CCc3ccccc3)C(=O)[C@H](CC(C)C)N2C(=O)[C@@H]1CC(C)C] | (3S,6S,9aR)-3,6-diisobutyl-2-(4- methylpentanoyl)-8-phenethylhexahydro- 4H-pyrazino[1,2-a]pyrazine-4,7(6H)-dione | 483.7 | 484.3 |
| 35 | 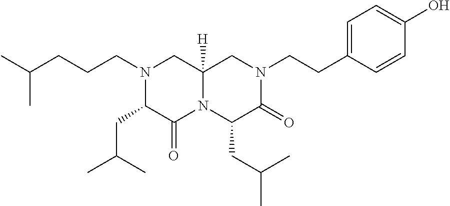 [SMILES: CC(C)CCCN1C[C@H]2CN(CCc3ccc(O)cc3)C(=O)[C@H](CC(C)C)N2C(=O)[C@@H]1CC(C)C] | (3S,6S,9aS)-8-(4-hydroxyphenethyl)-3,6- diisobutyl-2-(4-methylpentyl)hexahydro- 4H-pyrazino[1,2-a]pyrazine-4,7(6H)-dione | 485.7 | 486.4 |
| 36 | 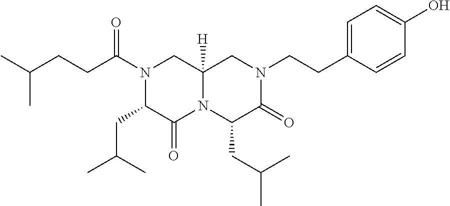 [SMILES: CC(C)CCC(=O)N1C[C@H]2CN(CCc3ccc(O)cc3)C(=O)[C@H](CC(C)C)N2C(=O)[C@@H]1CC(C)C] | (3S,6S,9aR)-8-(4-hydroxyphenethyl)-3,6- diisobutyl-2-(4- methylpentanoyl)hexahydro-4H- pyrazino[1,2-a]pyrazine-4,7(6H)-dione | 499.7 | 500.3 |
| 37 | 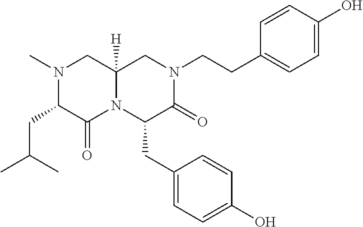 [SMILES: CC(C)C[C@H]1C(=O)N2[C@H](CN(CCc3ccc(O)cc3)C(=O)[C@@H]2Cc2ccc(O)cc2)CN1C] | (3S,6S,9aS)-6-(4-hydroxybenzyl)-8-(4- hydroxyphenethyl)-3-isobutyl-2- methylhexahydro-4H-pyrazino[1,2- a]pyrazine-4,7(6H)-dione | 465.6 | 466.2 |
| 38 | 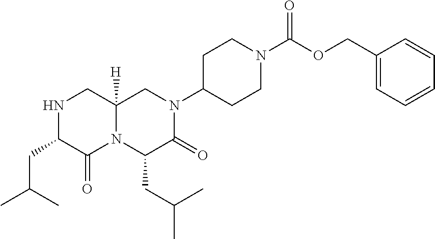 [SMILES: CC(C)C[C@@H]1NC[C@H]2CN(C3CCN(C(=O)OCc4ccccc4)CC3)C(=O)[C@H](CC(C)C)N2C1=O] | benzyl 4-((4S,7S,9aS)-4,7-diisobutyl-3,6- dioxooctahydro-2H-pyrazino[1,2- a]pyrazin-2-yl)piperidine-1-carboxylate | 498.7 | 499.3 |
| 39 | 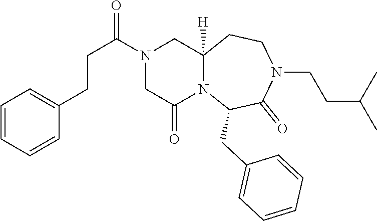 [SMILES: CC(C)CCN1CC[C@@H]2CN(C(=O)CCc3ccccc3)CC(=O)N2[C@@H](Cc2ccccc2)C1=O] | (6S,10aR)-6-benzyl-8-isopentyl-2-(3- phenylpropanyl)hexahydropyrazino[1,2- d][1,4]diazepine-4,7(1H,6H)-dione | 475.6 | 476.3 |
| 40 | 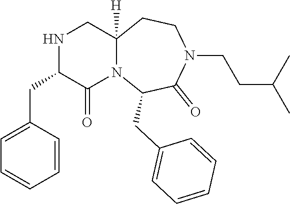 [SMILES: CC(C)CCN1CC[C@@H]2CN[C@@H](Cc3ccccc3)C(=O)N2[C@@H](Cc2ccccc2)C1=O] | (3S,6S,10aR)-3,6-dibenzyl-8- isopentylhexahydropyrazino[1,2- d][1,4]diazepine-4,7(1H,6H)-dione | 433.6 | 434.3 |
| 41 | 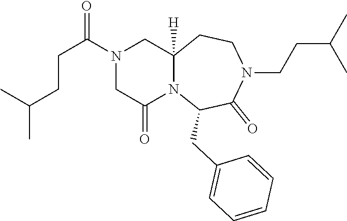 [SMILES: CC(C)CCC(=O)N1CC(=O)N2[C@H](CCN(CCC(C)C)C(=O)[C@@H]2Cc2ccccc2)C1] | (6S,10aR)-6-benzyl-8-isopentyl-2-(4- methylpentanoyl)hexahydropyrazino[1,2- d][1,4]diazepine-4,7(1H,6H)-dione | 441.6 | 442.4 |
| 42 | 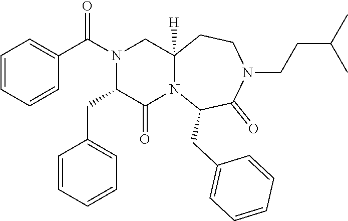 [SMILES: CC(C)CCN1CC[C@@H]2CN(C(=O)c3ccccc3)[C@@H](Cc3ccccc3)C(=O)N2[C@@H](Cc2ccccc2)C1=O] | (3S,6S,10aR)-2-benzyl-3,6-dibenzyl-8- isopentylhexahydropyrazino[1,2- d][1,4]diazepine-4,7(1H,6H)-dione | 537.7 | 538.4 |
| 43 | 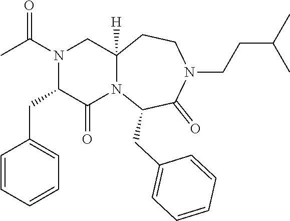 [SMILES: CC(=O)N1C[C@H]2CCN(CCC(C)C)C(=O)[C@H](Cc3ccccc3)N2C(=O)[C@@H]1Cc1ccccc1] | (3S,6S,10aR)-2-acetyl-3,6-dibenzyl-8- isopentylhexahydropyrazino[1,2- d][1,4]diazepine-4,7(1H,6H)-dione | 475.6 | 476.4 |

### Experimental Example: Notch Assay

We used the CellSensor T-REX™ NICD CSL-bla HeLa cell line, which was engineered by lentiviral transduction of Hela cells with a Notch response element driving beta-lactamase reporter gene expression (CSL-bla) along with DOX (doxycycline)-inducible NICD (Notch intracellular domain) constructs. Addition of DOX to these cells allows for regulated NICD transcription factor expression and subsequent beta-lactamase expression.

Specifically, the following protocol was used.

### Notch Assay Protocol

1. Add 40 nL/well of working concentration of 1000× compound to 384-well assay plate with Echo.
2. Plate Cells: 35 μl/well of HeLa/T-REX™ NICD CSL-bla cells at 10³ cells/well complete medium into 384-well plate.
3. Add 5 μl 8X Doxycycline in Assay Medium to the treated wells, and 5 μl Assay Medium to the Untreated and cell free control wells.
4. Incubate the assay plate in a humidified 37° C./5% CO₂ incubator for 16-20 hr.
5. Preparation of 6X LiveBLAzer™-FRET B/G Substrate (CCF4-AM) Mixture according to the manual and cell loading should be done in the absence of direct strong lighting.
6. Remove assay plate from the humidified 37° C./5% CO₂ incubator. Add 8 μl of 6X Substrate Mixture from Step5 to each well. Cover the plate to protect it from light and evaporation. Incubate at room temperature for 4 hr.
7. Reading an Assay Plate using the following filter selections:

TABLE 8

| Envision setting | Scan 1 | Scan 2 |
|---|---|---|
| Purpose | Measure fluorescence | Measure fluorescence |
| | in the blue channel | in the green channel |
| Excitation filter | 409/20 nm | 409/20 nm |
| Emission filter | 460/40 nm | 530/30 nm |

8. Data analysis: Use the assay plate layout to identify the location of the Cell-free wells. These control wells are used for background subtraction. Determine the average emission from the Cell-free wells at both 460 nm (Average Blue Background) and 530 nm (Average Green Background). Subtract the Average Blue Background (data collected at 460 nm) from all of the blue emission data. Subtract the Average Green Background (data collected at 530 nm) from all of the green emission data. Calculate the Blue/Green Emission Ratio for each well, by dividing the background-subtracted blue emission values by the background-subtracted green emission values.

The results are shown in Table 9.

TABLE 9

| Example Nos. | activity |
|---|---|
| 5, 6, 8, 9, 13, 16, 21, 34, 35, 36, 39, 42 | A |
| 7, 11, 14, 17, 19, 20, 26, 33, 41, 43 | B |
| 1, 4, 10, 12, 15, 18, 22, 23, 24, 25, 27, 28, | C |
| 29, 30, 31, 32, 37, 40 | |

activity A: 100-75%,
activity B: 74.9-50%,
activity C: 49.9-25%.

### Industrial Applicability

The compound of the present invention inhibits Notch signal transduction, and thus can be used for treating diseases involving Notch signal transduction.

Although only some exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention.

This application is based on U.S. provisional patent application No. 63/139,443 (filing date: Jan. 20, 2021) filed in US, the contents of which are incorporated in full herein.

1. A compound represented by the following formula (I): 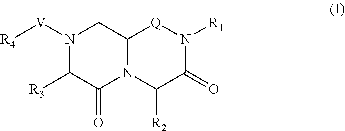
wherein
R₁ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl;
R₂ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl;
R₃ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl;
Q is —CH₂— or —CH₂CH₂—;
V is a bond, —CO—, —SO₂—, —NHCO—, or —OCO—; and
R₄ is hydrogen, optionally substituted alkyl, optionally substituted alkenyl, optionally substituted alkynyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl, or optionally substituted heterocycloalkylalkyl,
or a pharmaceutically acceptable salt thereof.

2. The compound according to claim 1, which is represented by the following formula (II): 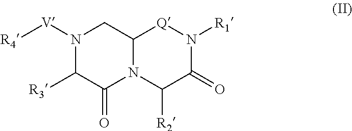
wherein
R₁′ is hydrogen, optionally substituted alkyl, optionally substituted heterocycloalkyl, or optionally substituted arylalkyl;
R₂′ is optionally substituted alkyl, or optionally substituted arylalkyl;
R₃′ is hydrogen, optionally substituted alkyl, or optionally substituted arylalkyl;
Q′ is —CH₂— or —CH₂CH₂—;
V′ is a bond, or —CO—; and
R₄′ is hydrogen, optionally substituted alkyl, optionally substituted aryl, or optionally substituted arylalkyl,
or a pharmaceutically acceptable salt thereof.

3. A pharmaceutical composition comprising an effective amount of compound according to claim 1 or a pharmaceutically acceptable salt thereof, and optionally a pharmaceutically acceptable carrier or diluent.

4. (canceled)

5. A method of treating or preventing a disease involving Notch signal transduction comprising administering to a subject in need thereof a compound according to claim 1 or a pharmaceutically acceptable salt thereof, in an amount effective to treat or prevent the disease.

6. (canceled)

7. (canceled)
